SEQUENCE LISTING (14 sequences)

SEQ ID NO: 1 - DNA, 491 bp, Homo sapiens
gcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggtaagaaactatgtgaaaactttttgattatgcatatgaacccttcacactacccaaattatatatttggctccatattcaatcggttagtctacatatatttatgtttcctctatgggtaagctactgtgaatg

SEQ ID NO: 2 - DNA, 20 bp, Homo sapiens
gcagagtacctgaaacagga

SEQ ID NO: 3 - DNA, 20 bp, Homo sapiens
cattcacagtagcttaccca

SEQ ID NO: 4 - DNA, 15 bp, Homo sapiens
caccaaagatgatat

SEQ ID NO: 5 - DNA, 15 bp, Homo sapiens
caccaaagacgatat

SEQ ID NO: 6 - DNA, 15 bp, Homo sapiens
caccacagatgatat

SEQ ID NO: 7 - DNA, 15 bp, Homo sapiens
caccagagatgatat

SEQ ID NO: 8 - DNA, 15 bp, Homo sapiens
aacaccaatgatatt

SEQ ID NO: 9 - DNA, 488 bp, Homo sapiens
gcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccagacttcacttctaatgatgattatgggagaactggagccttcagagggtaaaattaagcacagtggaagaatttcattctgttctcagttttcctggattatgcctggcaccattaaagaaaatatcattggtgtttcctatgatgaatatagatacagaagcgtcatcaaagcatgccaactagaagaggtaagaaactatgtgaaaactttttgattatgcatatgaacccttcacactacccaaattatatatttggctccatattcaatcggttagtctacatatatttatgtttcctctatgggtaagctactgtgaatg

SEQ ID NO: 10 - DNA, 488 bp, Homo sapiens; identical to SEQ ID NO: 9

SEQ ID NO: 11 - DNA, 15 bp, Homo sapiens
aatatcattggtgtt

SEQ ID NO: 12 - DNA, 50 bp, Homo sapiens
tggcaccattaaagaaaatatcatctttggtgtttcctatgatgaatata

SEQ ID NO: 13 - DNA, 15 bp, Homo sapiens
taccaaagatgatat

SEQ ID NO: 14 - DNA, 15 bp, Homo sapiens
caccaaagatgatac

(19) United States
(12) Patent Application Publication
(10) Pub. No.: US 2004/0180345 A1
(43) Pub. Date: Sep. 16, 2004

(54) Pre-incubation method to improve signal/noise ratio of nucleic acid assays

(75) Inventors: Glen H. Erikson, Providenciales (TC); Jasmine I. Daksis, Richmond Hill, CA (US)

Correspondence Address: CAESAR, RIVISE, BERNSTEIN,, COHEN & POKOTILOW, LTD., 11TH FLOOR, SEVEN PENN CENTER, PHILADELPHIA, PA, 19103-2212, US

(73) Assignee: Ingeneus Corporation, Bridgetown (BB)

(21) Appl. No.: 10/389,033

(22) Filed: Mar. 14, 2003

(51) Int. Cl. C12Q 1/68

(52) U.S. Cl. 435/6

(57) ABSTRACT

A method for assaying a nucleobase-containing target with a nucleobase-containing probe, wherein: (a) the target is pre-incubated with at least one target incubation agent prior to being mixed with the probe; and/or (b) the probe is pre-incubated with at least one probe incubation agent prior to being mixed with the target to form the hybridization mixture. The pre-incubation enhances the signal to noise ratio of the assay. The pre-incubation medium and/or the hybridization medium can be pretreated with electric voltage. A kit for performing the method includes the probe, a label adapted to emit the signal, and at least one target incubation agent and/or probe incubation agent.

### BACKGROUND OF THE INVENTION

1. Field of Invention

The invention relates to nucleobase binding in complexes, such as duplexes, triplexes and quadruplexes, and more particularly to a method for detecting such complexes with an improved signal to noise ratio.

2. Description of Related Art

The inventors have previously disclosed Watson-Crick quadruplexes and other non-canonical quadruplexes, triplexes and duplexes in, e.g., U.S. Patent Application 20020031775 A1, published Mar. 14, 2002. That application provides ample guidance regarding the selection of appropriate hybridization conditions to obtain any of the various multiplexes disclosed, including parallel or antiparallel duplexes, triplexes or quadruplexes binding in the homologous or Watson-Crick motif. See also U.S. Pat. No. 6,420,115 to Erikson et al. and U.S. Pat. No. 6,403,313 to Daksis et al.

Despite the foregoing developments, it is desired to improve the sensitivity of all existing detection methods. It is further desired to increase the signal/noise ratio of all detection methods.

All references cited herein are incorporated herein by reference in their entireties.

### BRIEF SUMMARY OF THE INVENTION

The invention provides a method for assaying a target, said method comprising:

providing a target composition comprising the target in a target medium, wherein the target contains a target sequence of nucleic acids or nucleic acid analogues;

providing a probe composition comprising a probe in a probe medium, wherein the probe contains a probe sequence of nucleic acids or nucleic acid analogues;

providing a hybridization mixture comprising the target composition and the probe composition;

incubating the hybridization mixture for an incubation period effective to bind the target sequence to the probe sequence to provide a complex, wherein the probe sequence is bonded to the target sequence by Watson-Crick complementary base interaction or by homologous base interaction; and

detecting a signal correlated with a binding affinity of the probe for the target to assay the target,

wherein: (a) the target composition further comprises at least one target incubation agent and the target composition is incubated prior to being provided in the hybridization mixture, such that discrimination of the signal from background signals is enhanced; and/or (b) the probe composition further comprises at least one probe incubation agent and the probe composition is incubated prior to being provided in the hybridization mixture, such that discrimination of the signal from background signals is enhanced

It is further provided that any of the target medium, probe medium, or hybridization mixture can be pre-treated with electric voltage prior to or during any of the incubations of same.

Also provided is a kit for performing the method of the invention, wherein the kit comprises the probe, a label adapted to emit the signal, and at least one of the target incubation agent and the probe incubation agent.

### DETAILED DESCRIPTION OF THE INVENTION

The invention is useful for enhancing the sensitivity of any method for assaying interaction between nucleobase-containing sequences. Thus, the invention not only improves upon the assay methods previously disclosed by the inventors in their patents and patent applications referenced above, it will also enhance the sensitivity of more conventional assays, including those based on canonical interactions between nucleobase-containing probes and targets to form antiparallel Watson-Crick duplexes.

The invention flows from our discovery that pre-incubation of the probe with a probe incubation agent and/or the target with a target incubation agent can increase discrimination of the signal to be detected from background signals (i.e., interference or background noise) by: (a) increasing binding affinity or signal strength of perfectly matched target and probe; and/or (b) decreasing binding affinity or signal strength of mismatched target and probe. The term “pre-incubation” is intended to denote a step or steps that precede mixing of the probe and the target (i.e., incubation). Pre-incubation can immediately precede incubation, or can precede incubation and one or more other steps that also precede incubation.

The precise duration of pre-incubation may vary according to the nature of the probe, target and incubation agents, but can be determined by routine experimentation using the present disclosure as a guide. In certain embodiments, wherein the probe is not pre-incubated, the target is pre-incubated with one or more target incubation agents for about 5 minutes to about 25 minutes, preferably about 15 minutes. In certain other embodiments, wherein the target is not pre-incubated, the probe is pre-incubated with one or more probe incubation agents for about 1 hour to about 3 hours, preferably about 2 hours. In still other embodiments, wherein both the target and the probe are pre-incubated with their respective incubation agents, the target is pre-incubated with the target incubation agent for about 5 minutes to about 25 minutes, preferably about 15 minutes, and the probe is pre-incubated with the probe incubation agent for about 15 minutes to about 3 hours. Pre-incubation can follow the addition of medium, which can be treated by electric voltage.

The medium can be pretreated with electric voltage prior to being added to the hybridization mixture. Such pretreatment can further enhance specific binding affinity of the probe for the target and/or enhance the specificity of the assay. Additional information regarding pretreatment is disclosed in U.S. patent application Ser. No. 10/189,211, filed Jul. 3, 2002.

A target incubation agent can be the same as or different from a probe incubation agent. An agent can, in certain embodiments, be independently selected from the group consisting of intercalating agents and cations, such as metal cations. Preferred intercalating agents include but are not limited to YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, cyanine monomers, ethidium bromide, ethidium homodimer-1, ethidium homodimer-2, ethidium derivatives, acridine, acridine orange, acridine derivatives, ethidium-acridine heterodimer, ethidium monoazide, propidium iodide, SYTO dyes, SYBR Green 1, SYBR dyes, Pico Green, SYTOX dyes and 7-aminoactinomycin D, with YOYO-1 being most preferred. Other preferred agents include cations including but not limited to Na⁺, K⁺, Mg⁺², Mn⁺², spermidine and spermine, with Na⁺ being most preferred.

The identity and amounts of probe and target incubation agents may vary according to the nature of the probe and target of the assay and the circumstances of the hybridization mixture and the acquisition of one or more signals therefrom, but can be determined by routine experimentation using the present disclosure as a guide. In certain embodiments, the probe incubation agent is provided in the probe composition at a concentration of about 20 nM to about 100 nM. In certain embodiments, the target incubation agent is provided in the target composition at a concentration of about 50 nM to about 100 mM, preferably about 50 nM to about 100 nM.

In certain embodiments, the probe incubation agent (PIA) is provided in the probe composition at a PIA:probe ratio of 0.25:1 to 2000:1. In certain embodiments, the target incubation agent (TTA) is provided in the target composition at a TIA:target ratio of 5:1 to 2,000,000:1. The PIA:probe ratio for metal cation PIAs is preferably 5:1 to 2000:1, more preferably 80:1 to 160:1. The PIA:probe ratio for PIAs other than metal cations is preferably 0.25:1 to 100:1, more preferably 1:1 to 10:1, with a ratio of 1.28:1 to 6.4:1 being most preferred for triplex formation and 1.28:1 to 9.6:1 being most preferred for duplex formation. The TIA:target ratio for metal cation TTAs is preferably 5:1 to 2,000,000:1, with 80:1 to 160,000:1 being most preferred for triplex formation. The TIA:target ratio for TIAs other than metal cations is preferably 5:1 to 1280:1, with 80:1 to 160:1 being most preferred for triplex formation and 20:1 to 320:1 being most preferred for duplex formation.

The sensitivity enhancing effect of the present invention can be used with canonical and non-canonical duplexes, triplexes and quadruplexes of nucleic acids and/or nucleic acid analogues, including the non-canonical duplexes, triplexes and quadruplexes disclosed in our earlier patents and patent applications, including U.S. Patent Application 20020031775 A1, published Mar. 14, 2002.

In general, the probe can be pre-incubated in a probe composition. The probe composition comprises the probe, the probe incubation agent and a probe medium. In certain preferred embodiments, electricity is applied to the probe medium before the probe is added. It has been found that such electrification further enhances the sensitivity of the assay and/or diminishes the amount of time necessary to achieve a desired enhancement otherwise achieved by pre-incubation of the probe and the use of probe incubation agents. In certain embodiments, it is preferred to apply a plurality of DC electric pulses having a voltage of about 9 volts each. Other conditions for effectively applying electric voltage to the probe medium can be determined through routine experimentation using the present disclosure as a guide.

The target is pre-incubated in a target composition. The target composition comprises the target, the target incubation agent and a target medium. In any hybridization mixture, the target medium and the probe medium can be the same or different, as can the probe incubation agents and the target incubation agents. Preferably, the target medium and the probe medium comprise any conventional medium known to be suitable for preserving nucleotides.

Likewise, the hybridization mixture can include any conventional medium known to be suitable for preserving nucleotides. See, e.g., Sambrook et al., “Molecular Cloning: A Lab Manual,” Vol. 2 (1989). For example, the medium can comprise nucleotides, water, buffers and standard salt concentrations. When divalent cations are used exclusively to promote triplex or quadruplex formation, chelators such as EDTA or EGTA should not be included in the reaction mixtures.

Specific binding between complementary bases occurs under a wide variety of conditions having variations in temperature, salt concentration, electrostatic strength, and buffer composition. Examples of these conditions and methods for applying them are known in the art. Our copending U.S. patent application Ser. No. 09/885,731, filed Jun. 20, 2001, discloses conditions particularly suited for use in triplex and quadruplex assays of the invention.

The inventive assay can be performed under conventional duplex hybridization conditions, under triplex hybridization conditions, under quadruplex hybridization conditions or under conditions of in situ hybridization. It is preferred that complexes be formed at a temperature of about 2° C. to about 55° C. for about two hours or less. In certain embodiments, the incubation period is preferably less than five minutes, even at room temperature. Longer reaction times are not required, but incubation for up to 24 hours in most cases, does not adversely affect the complexes.

The promoter in the hybridization medium is preferably an intercalating agent or a cation, as disclosed in U.S. Pat. No. 6,420,115 to Erikson et al. The intercalators are optionally fluorescent. The intercalating agent can be, e.g., a fluorophore, such as a member selected from the group consisting of YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, cyanine monomers, ethidium bromide, ethidium homodimer-1, ethidium homodimer-2, ethidium derivatives, acridine, acridine orange, acridine derivatives, ethidium-acridine heterodimer, ethidium monoazide, propidium iodide, SYTO dyes, SYBR Green 1, SYBR dyes, Pico Green, SYTOX dyes and 7-aminoactinomycin D.

Suitable cations for use in the hybridization medium include, e.g., monovalent cations, such as Na⁺ (preferably at a concentration of 40 mM to 200 mM), K⁺ (preferably at a concentration of 40 mM to 200 mM), and other alkali metal ions; divalent cations, such as alkaline earth metal ions (e.g., Mg⁺² and Ca⁺²) and divalent transition metal ions (e.g., Mn⁺², Ni⁺², Cd⁺², Co⁺² and Zn⁺²); and cations having a positive charge of at least three, such as Co(NH₃)₆⁺³, trivalent spermidine and tetravalent spermine. Mn⁺² is preferably provided at a concentration of 10 mM to 45 mM. Mg⁺² is preferably provided at a concentration of 10 mM to 45 mM. Ni⁺² is preferably provided at a concentration of about 20 mM. In embodiments, Mg⁺² and Mn⁺² are provided in combination at a concentration of 1 mM each, 2 mM each, 3 mM each . . . 40 mM each (i.e., 1-40 mM each).

The amount of cation added to the hybridization medium in which the complex forms depends on a number of factors, including the nature of the cation, the concentration of probe, the concentration of target, the presence of additional cations and the base content of the probe and target. The preferred cation concentrations and mixtures can routinely be discovered experimentally. For triplexes, it is preferred to add cation(s) to the medium in the following amounts: (a) 10 mM-30 mM Mn⁺²; (b) 10 nM-20 mM Mg 2; (c) 20 mM Ni⁺²; or (d) 1 mM-30 mM of each of Mn⁺² and Mg⁺². For quadruplexes, it is preferred to add cation(s) to the medium in the following amounts: (a) 10 mM-45 mM Mn⁺²; (b) 10 mM-45 mM Mg⁺²; or (c) 10 mM-40 mM of each of Mn⁺² and Mg⁺².

Although not required, other promoters include, e.g., single stranded binding proteins such as Rec A protein, T4 gene 32 protein, E. coli single stranded binding protein, major or minor nucleic acid groove binding proteins, viologen and additional intercalating substances such as actinomycin D, psoralen, and angelicin. Such facilitating reagents may prove useful in extreme operating conditions, for example, under abnormal pH levels or extremely high temperatures. Certain methods for providing complexes of the invention are conducted in the absence of protein promoters, such as Rec A and/or other recombination proteins.

The invention provides a rapid, sensitive, environmentally friendly, and safe method for assaying binding. The inventive assay can be used to, e.g., identify accessible regions in folded nucleotide sequences, to determine the number of mismatched base pairs in a hybridization complex, and to map genomes.

The inventive assay methods not only allow for detection of the presence of specific probe-target binding, but can also provide qualitative and quantitative information regarding the nature of interaction between a probe and target. Thus, the invention enables the practitioner to distinguish among a perfect match, a one base pair mismatch, a two base pair mismatch, a three base pair mismatch, a one base pair deletion, a two base pair deletion and a three base pair deletion arising between a sequence in the double-stranded probe or single-stranded probe and in a sequence in the double-stranded or single-stranded target.

Embodiments of the invention comprise calibrating the measured signal (e.g., optical, fluorescence, chemiluminescence, electrochemiluminescence, electrical or electromechanical properties) for a first probe-target mixture against the same type of signal exhibited by other probes combined with the same target, wherein each of the other probes differs from the first probe by at least one base.

A calibration curve can be generated, wherein the magnitude of the measured signal (e.g., fluorescent intensity) is a function of the binding affinity between the target and probe. As the binding affinity between the target and a plurality of different probes varies with the number of mismatched bases, the nature of the mismatch(es) (e.g., A:G vs. A:C vs. T:G vs. T:C, etc. in the W-C motif), the location of the mismatch(es) within the complex, etc., the assay of the invention can be used to sequence the target.

In embodiments, the signal measured can be the fluorescent intensity of a fluorophore included in the test sample. In such embodiments, the binding affinity between the probe and target can be directly or inversely correlated with the intensity, depending on whether the fluorophore signals hybridization through signal quenching or signal amplification. Under selected conditions, the fluorescent intensity generated by intercalating agents can be directly correlated with probe-target binding affinity, whereas the intensity of preferred embodiments employing a non-intercalating fluorophore covalently bound to the probe can be inversely correlated with probe-target binding affinity. The fluorescent intensity decreases for non-intercalating fluorophores as the extent of matching (e.g., the amount of matches vs. mismatches and/or the types of mismatches) between the probe and target increases, preferably over a range inclusive of 0-2 mismatches and/or deletions, more preferably over a range inclusive of 0-3 mismatches and/or deletions.

The invention enables quantifying the binding affinity between probe and target. Such information can be valuable for a variety of uses, including designing antisense or antigene drugs with optimized binding characteristics.

The assay of the invention is preferably homogeneous. The assay can be conducted without separating free probe and free target from the hybridization complex prior to detecting the magnitude of the measured signal. The assay does not require a gel separation step, thereby allowing a great increase in testing throughput. Quantitative analyses are simple and accurate. Consequently the binding assay saves a lot of time and expense, and can be easily automated. Furthermore, it enables binding variables such as buffer, pH, ionic concentration, temperature, incubation period, relative concentrations of probe and target sequences, intercalator concentration, length of target sequences, length of probe sequences, and possible cofactor (i.e., promoter) requirements to be rapidly determined.

The assay can be conducted in, e.g., a solution within a well or microchannel, on an impermeable surface, on a semi-permeable membrane, or on a biochip, wherein at least one of the probe and the target are bound to the support. In certain embodiments, the target is provided in the hybridization medium before the probe, and the probe is provided in dehydrated form prior to rehydration by contact with the hybridization medium.

In certain embodiments, the inventive assay is conducted without providing a signal quenching agent on the target or on the probe.

In certain embodiments of the invention, the target does not need to be denatured prior to assaying. It is surprising that the inventors have been able to specifically assay heteropolymeric triplexes and quadruplexes, wherein the interaction between the probes and targets is based on Watson-Crick or homologous base interaction (at least in the sense that A binds to T (or U, in the case of RNA) and G binds to C), rather than the very limited Hoogsteen model of complex hybridization of, e.g., U.S. Pat. No. 5,888,739 to Pitner et al.

Suitable targets are preferably 8 to 3.3×10⁹ base pairs long, and can be single or double-stranded.

Probes of the invention are preferably 2 to 75 bases long (more preferably 5 to 30 bases long), and can be single or double-stranded. Thus, suitable probes for use in the inventive assay include, e.g., ssDNA, RNA, ssPNA, LNA, dsDNA, dsRNA, DNA:RNA hybrids, dsPNA, PNA:DNA hybrids and other single and double-stranded nucleic acids and nucleic acid analogues having uncharged, partially-charged, sugar phosphate and/or peptide backbones. The length of the probe can be selected to match the length of the target.

The instant invention does not require the use of radioactive probes, which are hazardous, tedious and time-consuming to use, and need to be constantly regenerated. Probes of the invention are preferably safe to use and stable for years. Accordingly, probes can be made or ordered in large quantities and stored.

The complex is preferably detected by a change in at least one label. The at least one label can be attached to the probe and/or the target, and/or can be free in the test medium. The at least one label can comprise at least two moieties.

The label is preferably at least one member selected from the group consisting of a spin label, a fluorophore, a chromophore, a chemiluminescent agent, an electro-chemiluminescent agent, a radioisotope, an enzyme, a hapten, an antibody and a labeled antibody. Preferably, the complex is detected by at least one emission from the label or by monitoring an electronic characteristic of the complex.

The labeled antibody can be, e.g., a labeled anti-nucleic acid/nucleic acid antibody, which can be labeled with a detectable moiety selected from the group consisting of a fluorophore, a chromophore, a spin label, a radioisotope, an enzyme, a hapten, a chemiluminescent agent and an electro-chemiluminescent agent.

The complex can be detected under at least one varied condition, such as disclosed in U.S. Pat. No. 6,265,170 to Picard et al. Suitable varied conditions include, e.g., (a) a change in nonaqueous components of the test medium, (b) a change in a pH of the test medium, (c) a change in a salt concentration of the test medium, (d) a change of an organic solvent content of the test medium, (e) a change in a formamide content of the test medium, (f) a change in a temperature of the test medium, and (g) a change in chaotropic salt concentration in the test medium. In addition, the varied condition can be the application of a stimulus, such as, e.g., electric current (DC and/or AC), photon radiation (e.g., laser light), or electromagnetic force. The stimulus can be applied constantly or pulsed. Detection can be accomplished through the use of a single varied condition, or through a combination of conditions varied serially.

The response of a characteristic of the complex in the test medium to the varied condition or stimulus can be monitored to detect the complex. The characteristic can be, e.g., electrical conductance or Q (a resonant structure of a transmission line or changes in phase or amplitude of a signal propagated in the transmission line in the test medium).

In embodiments, the detection method comprises: (a) detecting a signal from a label, wherein the signal is correlated to a binding affinity between said probe and said target; (b) varying a condition of a test medium; (c) detecting a subsequent signal; and (d) comparing the signal and the subsequent signal. The varying and the detecting can be repeated at least once or performed only once.

The label is preferably a fluorophore. Both intercalating and non-intercalating fluorophores are suitable for use in the invention. The fluorophore can be free in solution, covalently bound to the probe and/or covalently bound to the target. When the fluorophore is covalently bound to the probe, it is preferably bound to the probe at either end. Preferred fluorescent markers include biotin, rhodamine, acridine and fluorescein, and other markers that fluoresce when irradiated with exciting energy. Suitable non-intercalating fluorophores include, e.g., alexa dyes, BODIPY dyes, biotin conjugates, thiol reactive probes, fluorescein and its derivatives (including the “caged probes”), Oregon Green, Rhodamine Green and QSY dyes (which quench the fluorescence of visible light excited fluorophores).

The excitation wavelength is selected (by routine experimentation and/or conventional knowledge) to correspond to this excitation maximum for the fluorophore being used, and is preferably 200 to 1000 nm. Fluorophores are preferably selected to have an emission wavelength of 200 to 1000 nm. In preferred embodiments, a suitably powered argon ion laser is used to irradiate the fluorophore with light having a wavelength in a range of 400 to 540 nm, and fluorescent emission is detected in a range of 500 to 750 nm. The hybridization mixture is preferably irradiated with energy of about 25-150 W/cm², more preferably 80 W/cm².

The assay of the invention can be performed over a wide variety of temperatures, such as, e.g., from about 2 to about 60° C. Certain prior art assays require elevated temperatures, adding cost and delay to the assay. On the other hand, the invention can be conducted at room temperature or below (e.g., at a temperature below 25° C.).

The reliability of the invention is independent of guanine and cytosine content in either the probe or the target. In the traditional W-C motif, since G:C base pairs form three hydrogen bonds, while A:T base pairs form only two hydrogen bonds, target and probe sequences with a higher G or C content are more stable, possessing higher melting temperatures. Consequently, base pair mismatches that increase the GC content of the hybridized probe and target region above that present in perfectly matched hybrids may offset the binding weakness associated with a mismatched probe.

The inventive assay is extremely sensitive, thereby obviating the need to conduct PCR amplification of the target. For example, it is possible to assay a test sample having a volume of about 20 microliters, which contains about 10 femtomoles of target and about 10 femtomoles of probe. Embodiments of the invention are sensitive enough to assay targets at a concentration of 5×10⁻⁹ M, preferably at a concentration of not more than 5×10⁻¹⁰ M. Embodiments of the invention are sensitive enough to employ probes at a concentration of 5×10⁻⁹ M, preferably at a concentration of not more than 5×10⁻¹⁰ M. It should go without saying that the foregoing values are not intended to suggest that the method cannot detect higher concentrations.

The ratio of probe to target is preferably about 1:1 to about 1000:1.

Unlike certain prior art assays, the invention not only detects the presence of hybridization (i.e., binding), but also provides qualitative and quantitative information regarding the nature of binding between a probe and target. Thus, the invention enables the practitioner to: (a) detect the presence of the target in the test medium; (b) detect allelic or heterozygous variance in the target; (c) quantitate the target; (d) detect an extent of complementarity (in the case of binding in the W-C motif) or homologousness (in the case of binding in the homologous motif) between the probe and the target; and (e) detect haplotypes.

We have noticed that duplexes which complex parallel strands of nucleic acid containing complementary base sequences bind to form triplexes at a different rate and bind as a culmination of a very different process than do bases in a double helix formed by nucleic acid strands of opposite directionality. Strands of opposite directionality (i.e., antiparallel strands) readily present regularly spaced bases in a planar orientation to the bases opposite with minimal backbone distortion.

The various complexes formed upon practicing the methods of the invention comprise a probe containing a heteropolymeric probe sequence of nucleobases and/or nucleobase analogues, and a target containing a heteropolymeric target sequence of nucleobases and/or nucleobase analogues. The complex can be synthetic or purified in that at least one of either the probe or the target is synthetic or purified. The backbone of the probe can be a deoxyribose phosphate backbone such as in DNA, or a peptide-like backbone such as in PNA, or is of some other uncharged or partially charged (negatively or positively) moieties.

In certain embodiments, the probe and target are single-stranded and the complex is a duplex. When said probe and target are a duplex they can have parallel or antiparallel directionality with W-C complementary or homologous binding.

In other embodiments, either the probe or the target is single-stranded and the other of said probe or target is double-stranded and the resulting complex is a triplex. This complex can be free of PNA.

In certain embodiments, the triplex contains a heteropolymeric probe sequence parallel to a heteropolymeric target sequence, wherein the heteropolymeric probe sequence is bonded to the heteropolymeric target sequence by homologous base binding or Watson-Crick complementary base binding. In certain other embodiments, the heteropolymeric probe sequence is antiparallel to the heteropolymeric target sequence and the heteropolymeric probe sequence is bonded to the heteropolymeric target sequence by homologous base binding or Watson-Crick complementary base binding.

In certain embodiments of the triplex complex, the target includes a first strand containing a heteropolymeric target sequence and a second strand containing a second heteropolymeric target sequence that is Watson-Crick complementary and antiparallel to the first heteropolymeric target sequence. The heteropolymeric probe sequence is bonded to the first heteropolymeric target sequence by homologous base bonding and is also bonded to the second heteropolymeric target sequence by Watson-Crick complementary base bonding.

In certain other embodiments of the triplex complex, the target includes a first strand containing a heteropolymeric target sequence and a second strand containing a second heteropolymeric target sequence that is Watson-Crick complementary and antiparallel to the first heteropolymeric target sequence. The heteropolymeric probe sequence is bonded to the first heteropolymeric target sequence by Watson-Crick complementary base bonding and is also bonded to the second heteropolymeric target sequence by homologous base bonding.

In certain embodiments, the probe and the target are double-stranded and the resulting complex is a quadruplex. This complex can be free of PNA.

In certain embodiments, the quadruplex contains a heteropolymeric probe sequence parallel or antiparallel to a heteropolymeric target sequence, wherein the heteropolymeric probe sequence is bonded to the heteropolymeric target sequence by homologous base binding or Watson-Crick complementary base binding. In such embodiments, the quadruplex complex contains a first probe strand containing said heteropolymeric probe sequence and a second probe strand containing a second heteropolymeric probe sequence that is complementary and antiparallel to the first probe sequence. The target includes a first target strand containing a heteropolymeric target sequence and a second target strand containing a second heteropolymeric target sequence that is complementary and antiparallel to the first.

In such quadruplex embodiments, the heteropolymeric probe sequence can bond to the heteropolymeric target sequence by Watson-Crick complementary or homologous base binding and the heteropolymeric probe sequence can optionally and additionally bond to the second heteropolymeric target sequence by homologous or Watson-Crick complementary base binding, respectively. Thus, when the heteropolymeric probe sequence bonds to the heteropolymeric target sequence by homologous base bonding, the heteropolymeric probe sequence optionally bonds to the second heteropolymeric target sequence by Watson-Crick complementary base bonding, and when the heteropolymeric probe sequence bonds to the heteropolymeric target sequence by Watson-Crick complementary base bonding, the heteropolymeric probe sequence optionally bonds to the second heteropolymeric target sequence by homologous base bonding.

The kit of the invention preferably comprises the probe, a label adapted to emit the signal, and at least one of the target incubation agent and the probe incubation agent. The target incubation agent can be the same as or different from the probe incubation agent. In the former case, the common incubation agent can therefore be provided in the kit in one or more portions (e.g., as a single container containing probe/target incubation agent for both purposes).

The label can be covalently bound to the probe in the kit, can covalently bond to the probe or target upon mixing with same, or can non-covalently associate (e.g., by intercalating) within complexes formed in the assay.

The invention will be illustrated in more detail with reference to the following Examples, but it should be understood that the present invention is not deemed to be limited thereto.

### EXAMPLES

### Example 1

Genomic dsDNA was extracted from a human blood sample using a QIAamp DNA blood purification kit (QIAGEN, Mississauga, Canada) as per manufacturer's instructions. A 491 bp dsDNA fragment (SEQ TD NO:1), corresponding to a clinically significant region of exon 10 of the cystic fibrosis gene, was PCR-amplified. Sequence for the 20-mer upper primer: 5′-GCA GAG TAC CTG AAA CAG GA-3′ (SEQ ID NO:2). Sequence for the 20-mer lower primer: 5′-CAT TCA CAG TAG CTT ACC CA-3′ (SEQ ID NO:3). 100 pmole of each primer was mixed with 1 μg genomic dsDNA in a 100 μl PCR reaction mix using a Taq PCR Master Mix Kit (QIAGEN, Mississauga, Canada). The following PCR cycle parameter was used: 1 cycle of 94° C.×5 min, 25 cycles of (93° C.×30 sec, 48° C.×30 sec, 72° C.×45 sec), 1 cycle of 72° C.×7 min. The size of the PCR fragment was confirmed by gel electrophoresis, but no purification to remove trace amounts of residual background primers or genomic DNA was performed. The concentration of the PCR-amplified 491 bp dsDNA target was determined by UV spectroscopy and a 1 pmole/μl stock solution was prepared.

Sequence for the sense strand of the wild-type PCR-amplified 491 bp dsDNA target (SEQ ID NO:1):

| | |
|---|---|
| gcagagtacc tgaaacagga agtattttaa atattttgaa tcaaatgagt taatagaatc | 60 |
| tttacaaata agaatataca cttctgctta ggatgataat tggaggcaag tgaatcctga | 120 |
| gcgtgatttg ataatgacct aataatgatg ggttttattt ccagacttca cttctaatga | 180 |
| tgattatggg agaactggag ccttcagagg gtaaaattaa gcacagtgga agaatttcat | 240 |
| tctgttctca gttttcctgg attatgcctg gcaccattaa agaaaatatc atctttggtg | 300 |
| tttcctatga tgaatataga tacagaagcg tcatcaaagc atgccaacta gaagaggtaa | 360 |
| gaaactatgt gaaaactttt tgattatgca tatgaaccct tcacactacc caaattatat | 420 |
| atttggctcc atattcaatc ggttagtcta catatattta tgtttcctct atgggtaagc | 480 |
| tactgtgaat g | 491 |

Antisense 15-mer ssDNA probe sequences, derived from exon 10 of the human cystic fibrosis gene (Nature 380, 207 (1996)) were synthesized on a DNA synthesizer (Expedite 8909, PerSeptive Biosystems), cartridge purified and dissolved in ddH₂O at a concentration of 1 pmole/μl.

Probe CF01 (SEQ ID NO:4) was a 15-mer ssDNA probe designed to be completely complementary to a 15 nucleotide segment of the sense strand of the wild-type PCR-amplified 491 bp dsDNA target (SEQ ID NO:1), overlapping amino acid positions 505 to 510 (Nature 380, 207 (1996)).

The sequence for probe CF01 (SEQ ID NO:4) was: 5′-CAC CAA AGA TGA TAT-3′.

Probes CF10, CF09 and CF08 were 15-mer mutant ssDNA probes identical in sequence to wild-type probe CF01, except for a one base mutation (underlined). The sequence for probe CF10 (SEQ ID NO:5) was: 5′-CAC CAA AGA CGA TAT-3′.

The sequence for probe CF09 (SEQ ID NO:6) was: 5′-CAC CAC AGA TGA TAT-3′.

The sequence for probe CF08 (SEQ ID NO:7) was: 5′-CAC CAG AGA TGA TAT-3′.

Probe CF508 was a 15-mer mutant ssDNA probe designed to be completely complementary to a 15 nucleotide segment of the sense strand of the wild-type 491 bp dsDNA target (SEQ ID NO:1), except for a consecutive three base deletion at amino acid positions 507 and 508 at which the wild-type antisense sequence AAG is deleted.

The sequence for probe CF508 (SEQ ID NO:8) was: 5′-AAC ACC AAT GAT ATT-3′.

The binding reaction mixture (80 μl) contained the following: 0.05 pmoles of PCR-amplified 491 bp dsDNA target, 1.25 pmoles of 15-mer ssDNA probe, 0.5×TBE and 100 nM of the DNA intercalator YOYO-1 (Molecular Probes, Eugene, Oreg., USA). All reaction mixtures described had a final volume of 80 μl. For Samples 2-6 of Table 1, all reagents were combined at the same time and incubated at room temperature (21° C.) for 5 minutes. For Samples 7-11 of Table 1, the 491 bp dsDNA target was pre-incubated in a volume of 76.35 μl containing 0.5×TBE buffer with 70 nM YOYO-1 for 15 min, with mixing steps at 7.5 min and 15 min, prior to the addition of a volume of 3.65 μl similarly buffered containing ssDNA probe and 30 nM YOYO-1. The reaction mixtures were then incubated at room temperature (21° C.) for 5 minutes. For Samples 12-16 of Table 1, the 491 bp dsDNA target was pre-incubated in a volume of 75.55 μl containing 0.5×TBE buffer with 60 nM YOYO-1 for 15 min, with mixing steps at 7.5 min and 15 min, prior to the addition of a volume of 4.45 μl similarly buffered containing ssDNA probe and 40 nM YOYO-1. The reaction mixtures were then incubated for 5 minutes. Following the final 5 min incubation the reaction mixtures were placed into a quartz cuvette, irradiated with a 10 mW argon ion laser beam having a wavelength of 488 nm and monitored for fluorescent emission immediately and then again after 90 min. The laser irradiation duration was 250 msec and delivered 80 W/cm² radiation. The emitted light was collected by CCD and documented by Ocean Optics software. The same detection equipment was used throughout these examples, unless otherwise indicated.

Perfectly matched DNA triplexes consisting of the 491 bp dsDNA (SEQ TD NO:1) and probe CF01 formed during a 5 min incubation with 100 nM YOYO-1 (Sample 4) allowed maximum intercalation of YOYO-1, yielding the highest fluorescent intensity after 5 min, which further increased after 90 min (Table 1). The fluorescent intensities for a one bp A-C mismatched dsDNA:ssDNA triplex (491 bp dsDNA+probe CF10) (Sample 6) were 60.1% and 100% lower after a 5 min and a 90 min incubation, respectively, than that of the perfectly matched triplex, when normalized for variations in different ssDNA probe fluorescence.

The 491 bp dsDNA target control pre-incubated with either 70 nM (Sample 7) or 60 nM YOYO-1 (Sample 12) showed a slightly reduced fluorescence than the dsDNA control incubated with 100 nM YOYO-1 (Sample 2). Similarly, the control wild-type and mutant ssDNA probes incubated with either 30 nM or 40 nM YOYO-1 showed reduced fluorescence compared to the same ssDNA probes incubated with 100 nM YOYO-1 (Table 1).

Pre-incubation of the 491 bp dsDNA target with either 70 nM or 60 nM YOYO-1 for 15 min prior to the addition of ssDNA probe and 30 nM or 40 nM YOYO-1, respectively, resulted in a reduction of formation (and hence fluorescence) of both the perfectly complementary DNA triplexes and the 1 bp A-C mismatched DNA triplexes (Table 1). The fluorescence emitted from the 1 bp A-C mismatched DNA triplexes relative to that of the perfectly matched DNA triplexes remained similar after a 5 min incubation of the reaction mixture, regardless of whether the dsDNA target was pre-incubated with YOYO-1 or not. Following a 90 min incubation of the reaction mixture, the fluorescence of the normalized 1 bp A-C mismatched DNA triplexes relative to that of the normalized perfectly matched DNA triplexes formed with reagents pre-incubated with a 60:40 split of YOYO-1 remained essentially the same as that observed following a 5 min reaction mixture incubation. However, the fluorescence of the normalized 1 bp A-C mismatched DNA triplexes, relative to that of the normalized perfectly matched DNA triplexes formed with a 70:30 split of YOYO-1, was reduced from 52.3% to 100% at the 90 min reaction mixture timepoint (Table 1). This preferential reduction of 1 bp mismatched DNA triplex formation of pre-incubated reagents with a 70:30 split of YOYO-1 was identical to that observed when 100 nM of YOYO-1 was added at once to the reaction mixture containing both dsDNA target and ssDNA probes.

Therefore a 15 min pre-incubation of 491 bp dsDNA target with either 70 nM or 60 nM YOYO-1 prior to addition of wild-type or mutant ssDNA probe with 30 nM or 40 nM YOYO-1, respectively, resulted in reduced target control and probe control fluorescence measured at 5 min, without a loss in specificity of DNA triplex formation between perfectly matched triplexes and 1 bp A-C mismatched triplexes.

All incubations or pre-incubations in this and all following examples were carried out at room temperature (21° C.)

### Example 2

Example 2 compares the effect of pre-incubating either dsDNA target or ssDNA probes with different concentrations of YOYO-1 prior to addition to the reaction mixture.

Table 2 shows the results when 0.05 pmoles of PCR-amplified 491 bp dsDNA target (SEQ ID NO:1) were pre-incubated in a volume of 76.35 μl containing 0.5×TBE buffer with 70 nM YOYO-1 for 15 min, with mixing steps at 7.5 min and 15 min, prior to the addition of 1.25 pmoles of wild-type or mutant ssDNA probe and 30 nM YOYO-1 in a volume of 3.65 μl. The 80 μl reaction mixtures were then incubated for 5 minutes, placed into a quartz cuvette, irradiated and monitored immediately for fluorescent emission.

The highest fluorescent emission intensity was achieved with dsDNA:ssDNA triplexes consisting of perfectly complementary sequences (491 bp dsDNA+probe CF01) (sample 4, Table 2). Incompletely complementary probe and target combinations generating a 1 bp A-C mismatch (491 bp dsDNA+probe CF10), a 1 bp T-C mismatch (491 bp dsDNA+probe CF09), a 1 bp T-G mismatch (491 bp dsDNA+probe CF08), and a 3 bp mismatch (491 bp dsDNA+probe CF508) resulted in fluorescent emission intensities that were 100%, 74.7%, 73.2% and 57.1% lower, respectively, than those observed with the perfectly matched sequences, when normalized for variations in different ssDNA probe fluorescence (Table 2). The control ssDNA probes, which were at 25-fold molar excess in concentration, showed similar and relatively high levels of fluorescence as compared to that emitted by the control 491 bp dsDNA target. These high levels of fluorescence of the ssDNA probes were likely a result of self-binding that resulted in the formation of parallel homologous complexes stabilized and signaled by YOYO-1. The variations in probe fluorescence among the probes were an expression of the affinity for self-binding characteristic of each probe sequence.

Table 3 shows the results when 1.25 pmoles of wild-type or mutant ssDNA probe were pre-incubated in a volume of 69.4 μl containing 0.5×TBE buffer with 30 nM YOYO-1 for 2 hr prior to the addition of 0.05 pmoles of PCR-amplified 491 bp dsDNA target (SFQ ID NO:1) and 70 nM YOYO-1 in a volume of 10.6 μl. The 80 μl reaction mixtures were then incubated for 5 minutes, placed into a quartz cuvette, irradiated and monitored immediately for fluorescent emission.

Pre-incubation of the control ssDNA probes with 30 nM YOYO-1 for 2 hr significantly reduced the fluorescent emission intensity of each ssDNA probe and consequently the fluorescence of each DNA triplex formed within 5 min, without altering the fluorescence of the control 491 bp dsDNA target (Table 3). The fluorescent emission intensities achieved by a 1 bp A-C mismatched DNA triplex (491 bp dsDNA+probe CF10), a 1 bp T-C mismatched DNA triplex (491 bp dsDNA+probe CF09), a 1 bp T-G mismatched DNA triplex (491 bp dsDNA+probe CF08), and a 3 bp mismatched DNA triplex (491 bp dsDNA+probe CF508) were 66.5%, 100%, 80.9% and 87.0% lower, respectively, than that obtained by the perfectly matched DNA triplex (491 bp dsDNA+probe CF01), when normalized against the respective levels of pre-incubated ssDNA probe control fluorescence (Table 3). In general, pre-incubation of the probe with 30 nM YOYO-1 for 2 hr prior to triplex formation resulted in slightly greater DNA triplex specificity than that achieved after pre-incubation of the dsDNA target with 70 nM YOYO-1 for 15 min prior to triplex formation (compare Table 2 and Table 3). The difference in DNA triplex specificity observed between the two binding protocols depended on the particular ssDNA probe sequence used to form the DNA triplex.

Variation of the pre-incubation period of the ssDNA probes with 30 nM YOYO-1 from 15 min to 3 hr revealed a progressive decline in probe fluorescence with time (data not shown). This was presumably due to a progressive decline of probe self-binding in the presence of YOYO-1 over time. Maximum reduction in probe fluorescence was observed following a 3 hr pre-incubation with 30 nM YOYO-1. However, a 3 hr pre-incubation also resulted in significant reduction in subsequent DNA triplex formation, with an observable decline in DNA triplex specificity (data not shown). The optimum pre-incubation period for ssDNA probes with 30 nM YOYO-1 was determined to be 2 hr, since such incubation significantly reduced probe alone fluorescence without subsequently sacrificing discrimination levels between perfectly matched DNA triplexes and bp mismatched DNA triplexes following the addition of the dsDNA target and 70 nM YOYO-1 (Table 3).

Table 4 shows the results when 1.25 pmoles of wild-type or mutant ssDNA probe were pre-incubated in a volume of 36.4 μl containing 0.5×TBE buffer with 30 nM YOYO-1 for 2 hr. At the 1.75 hr time-point, 0.05 pmoles of PCR-amplified 491 bp dsDNA target (SEQ ID NO:1) were pre-incubated in a volume of 43.6 μl containing 0.5×TBE buffer with 70 nM YOYO-1 for 15 min, with mixing steps at 7.5 min and 15 min. The pre-incubated targets were then mixed with the pre-incubated probes to generate an 80 μl reaction mixture, which was further incubated at room temperature for 5 minutes. The samples were placed into a quartz cuvette, irradiated and monitored immediately for fluorescent emission.

Pre-incubation of control ssDNA probes and control 491 bp dsDNA target with 30 nM YOYO-1 or 70 nM YOYO-1, respectively, resulted in diminished probe and target fluorescence as expected (Table 4). When both ssDNA probes and 491 bp dsDNA target were pre-incubated with 30 nM YOYO-1 or 70 nM YOYO-1, respectively, a further reduction in DNA triplex formation and fluorescence was observed as compared to that observed when only the ssDNA probes or dsDNA target were pre-incubated prior to addition to the reaction mixture (compare Tables 2, 3 and 4). When both ssDNA probes and 491 bp dsDNA target were pre-incubated with 30 nM YOYO-1 or 70 nM YOYO-1, respectively, the fluorescent emission intensities achieved by a 1 bp A-C mismatched DNA triplex (491 bp dsDNA+probe CF10), a 1 bp T-C mismatched DNA triplex (491 bp dsDNA+probe CF09), a 1 bp T-G mismatched DNA triplex (491 bp dsDNA+probe CF08), and a 3 bp mismatched DNA triplex (491 bp dsDNA+probe CF508) were 77.2%, 48.1%, 84.0% and 82.5% lower, respectively, than that obtained by the perfectly matched DNA triplex (491 bp dsDNA+probe CF01), when normalized for variations in different ssDNA probe fluorescence (Table 4). No significant improvement in DNA triplex specificity was achieved by pre-incubating both ssDNA probes and the dsDNA target with different concentrations of YOYO-1 prior to triplex formation. In one case, there was a noticeable loss in discrimination between the perfectly matched DNA triplex and the 1 bp T-C mismatched DNA triplex due to pre-incubation of both ssDNA probe and dsDNA target with YOYO-1, compared to the discrimination levels achieved following pre-incubation of only the ssDNA probe or the dsDNA target (compare Tables 2, 3 and 4).

For this reason, it is preferable to pre-incubate only one of two DNA binding partners, either the probe or the target with YOYO-1 prior to addition to the reaction mixture. Pre-incubation of the ssDNA probes with 30 nM YOYO-1 for 2 hr prior to addition of dsDNA target and 70 nM YOYO-1 appears to be the preferred protocol to significantly reduce ssDNA probe alone fluorescence while making possible a high degree of discrimination between perfectly matched DNA triplexes and mismatched DNA triplexes.

### Example 3

This example demonstrates how DNA triplex specificity is improved by the inclusion of selected concentrations of NaCl during the pre-incubation of dsDNA target with YOYO-1 prior to the addition of ssDNA probe with YOYO-1 to form reaction mixtures.

Table 5 shows the results when 0.05 pmoles of PCR-amplified 491 bp dsDNA target (SEQ ID NO:1) were pre-incubated in 0.5×TBE buffer with 70 nM YOYO-1 in the absence or presence of 50 nM, 75 nM or 50 mM NaCl for 15 min, with mixing steps at 7.5 min and 15 min, prior to the addition of 1.25 pmoles of wild-type or mutant ssDNA probe and 30 nM YOYO-1 to form reaction mixtures. The 80 μl reaction mixtures were then incubated for 5 minutes, placed into a quartz cuvette, irradiated and monitored immediately for fluorescent emission.

In the absence of NaCl, pre-incubation of the 491 bp dsDNA target with 70 nM YOYO-1 for 15 min resulted in a 40.0% decrease in fluorescence for the normalized 1 bp T-C mismatched DNA triplex (491 bp dsDNA+probe CF09) compared to that achieved with the normalized perfectly matched DNA triplex (491 bp dsDNA+probe CF01) (Table 5). Inclusion of either 50 nM or 75 nM NaCl during pre-incubation of the 491 bp dsDNA target with 70 nM YOYO-1 for 15 min, as well as in the control ssDNA probe samples, resulted in small changes in dsDNA target fluorescence or ssDNA probe fluorescence, but greatly improved the specificity of DNA triplex formation. The level of discrimination between subsequent perfectly matched DNA triplex formation and 1 bp T-C mismatched DNA triplex formation was increased from 40.0% (in the absence of NaCl during dsDNA target pre-incubation) to 58.6% and 100% when the dsDNA target was pre-incubated in the presence of 50 nM NaCl and 75 nM NaCl, respectively (Table 5).

Inclusion of 50 mM NaCl during pre-incubation of the 491 bp dsDNA target with 70 nM YOYO-1 for 15 min, resulted in a minimal decrease in fluorescence for the perfectly matched DNA triplex (491 bp dsDNA+probe CF01) but a dramatic loss in fluorescence for the 1 bp T-C mismatched DNA triplex (491 bp dsDNA+probe CF09) (Table 5). The difference in fluorescence between the perfectly matched DNA triplex and the 1 bp T-C mismatched DNA triplex increased to 91.1%. The presence of 50 mM NaCl in the control ssDNA probe samples had little effect on the fluorescence of the wild-type probe CF01, but severely reduced the fluorescence of the mutant probe CF09 (Table 5).

Therefore, the presence of selected concentrations of NaCl during the pre-incubation of dsDNA target with 70 nM YOYO-1 for 15 min, prior to the addition of wild-type or mutant ssDNA probe and 30 nM YOYO-1 to form a reaction mixture, preferentially increased perfectly matched DNA triplex formation and decreased 1 bp mismatched DNA triplex formation. As a consequence DNA triplex specificity was greatly improved.

### Example 4

This example examines how DNA triplex specificity can be influenced by the inclusion of low concentrations of NaCl during the pre-incubation of ssDNA probe with YOYO-1 prior to the addition of dsDNA target with YOYO-1 to form a reaction mixture.

Table 6 shows the results when 1.25 pmoles of wild-type or mutant ssDNA probe were pre-incubated in 0.5×TBE buffer with 30 nM YOYO-1 in the absence or presence of 50 nM, 75 nM or 100 nM NaCl for 2 hr prior to the addition of 0.05 pmoles of PCR-amplified 491 bp dsDNA target (SEQ ID NO:1) and 70 nM YOYO-1. The 80 μl reaction mixtures subsequently formed were then incubated for 5 minutes, placed into BD Biocoat Enhanced Recovery 384-well plates and irradiated with the GENEXUS ANALYZER 15 mW argon ion laser (available from Genetic Diagnostics, Inc., Toronto, Canada) having a wavelength of 488 nm and delivering 10 mW of laser light to the samples from the bottom of each well. Irradiation occurred at a sampling interval of 60 microns at settings of 1 hertz, 40% PMT and 10 μA/V sensitivity. These settings scan 2.7 msec/pixel. Fluorescent emission was monitored immediately.

Pre-incubation of the control ssDNA probes with 30 nM YOYO-1 for 2 hr significantly reduced the fluorescent emission intensity of each ssDNA probe, particularly the mutant ssDNA probe CF10 (Table 6). The addition of 50 nM to 100 nM NaCl during the pre-incubation of the probes with YOYO-1 marginally increased the fluorescence of each ssDNA probe.

In the absence of NaCl, pre-incubation of the ssDNA probes with 30 nM YOYO-1 for 2 hr resulted in a 48.6% decrease in fluorescence for the normalized 1 bp A-C mismatched DNA triplex (491 bp dsDNA+probe CF10) compared to that achieved with the normalized perfectly matched DNA triplex (491 bp dsDNA+probe CF01) (Table 6). Inclusion of either 50 nM or 75 nM NaCl during pre-incubation of the ssDNA probes with 30 nM YOYO-1 for 2 hr slightly increased both perfectly matched DNA triplex formation and 1 bp A-C mismatched DNA triplex formation. The level of discrimination between perfectly matched DNA triplex formation and 1 bp A-C mismatched DNA triplex formation was increased from 48.6% in the absence of NaCl to 54.0% and 53.7% in the presence of 50 nM NaCl and 75 nM NaCl, respectively (Table 6). The level of discrimination between perfectly matched DNA triplex formation and 1 bp A-C mismatched DNA triplex formation was reduced to 40.8% in the presence of 100 nM NaCl (Table 6).

Table 7 shows the results when 1.25 pmoles of wild-type or mutant ssDNA probe were pre-incubated in 0.5×TBE buffer in the absence or presence of 50 nM, 75 nM or 100 nM NaCl for 1 hr followed by a further incubation in the presence of 30 nM YOYO-1 for 2 hr prior to the addition of 0.05 pmoles of PCR-amplified 491 bp dsDNA target (SEQ ID NO:1) and 70 nM YOYO-1 to form reaction mixtures. The 80 μl reaction mixtures were then incubated for 5 minutes, placed into BD Biocoat Enhanced Recovery 384-well plates and irradiated with the GENEXUS ANALYZER 15 mW argon ion laser having a wavelength of 488 nm. 10 mW of laser light irradiates the samples from the bottom of each well. Irradiation occurred at a sampling interval of 60 microns at settings of 1 hertz, 40% PMT and 10 μA/V sensitivity. These settings scan 2.7 msec/pixel. Fluorescent emission was monitored immediately.

At these irradiation settings no fluorescence was observed from the ssDNA probes that were pre-incubated for a total of 3 hr during which 30 nM YOYO-1 had been present for 2 hr (Table 7). The extended incubation period also reduced DNA triplex formation. In the absence of NaCl, pre-incubation of the ssDNA probes in buffer for 1 hr followed by a further 2 hr incubation after addition of 30 nM YOYO-1 resulted in a 74.7% decrease in fluorescence for the 1 bp A-C mismatched DNA triplex (491 bp dsDNA+probe CF10) compared to that achieved with the perfectly matched DNA triplex (491 bp dsDNA+probe CFO) (Table 7). This extended incubation protocol greatly increased the specificity of DNA triplex formation (compare Tables 6 and 7).

The fluorescent emission intensities achieved by a 1 bp A-C mismatched DNA triplex (491 bp dsDNA+probe CF10) in the presence of 50 nM, 75 nM and 100 nM NaCl were 83.5%, 86.9% and 82.2% lower, respectively, than that obtained by the perfectly matched DNA triplexes (491 bp dsDNA+probe CF01) at these NaCl concentrations (Table 7). Inclusion of 50 nM, 75 nM or 100 nM NaCl during the 3 hr probe pre-incubation protocol preferentially increased perfectly matched DNA triplex formation with little effect on 1 bp A-C mismatched DNA triplex formation, thereby enhancing sensitivity and specificity of DNA triplex formation (Table 7).

### Example 5

This example demonstrates how DNA triplex specificity can be improved by the inclusion of 100 nM NaCl during the pre-incubation of dsDNA target with YOYO-1 when ssDNA probe with YOYO-1 has also been pre-incubated, but in the absence of NaCl.

Table 8 shows the results when 1.25 pmoles of wild-type or mutant ssDNA probe were pre-incubated in 0.5×TBE buffer with 30 nM YOYO-1 for 3 hr. During probe pre-incubation, 0.05 pmoles of PCR-amplified 491 bp dsDNA target (SEQ ID NO:1) were pre-incubated in separate tubes in 0.5×TBE buffer with 70 nM YOYO-1 in the absence or presence of 100 nM NaCl at room temperature (21° C.) for 15 min to 3h, calculated from the end of the probe pre-incubation period, with a mixing step at 15 min. The pre-incubated targets were then mixed with the pre-incubated probes to generate an 80 μl reaction mixture, which was further incubated for 5 minutes. The samples were placed into BD Biocoat Enhanced Recovery 384-well plates and irradiated with the GENEXUS ANALYZER 20 mW scanning solid state laser having a wavelength of 488 nm. 19 mW of laser light irradiates the samples from the bottom of each well. Irradiation occurred at a sampling interval of 60 microns at settings of 1 hertz, 40% PMT and 10 μA/V sensitivity. These settings scan 3.4 msec/pixel on the GENEXUS ANALYZER 20 mW solid state laser. Fluorescent emission was monitored immediately.

In the absence of NaCl, pre-incubation of the ssDNA probes with 30 nM YOYO-1 for 3 hr together with pre-incubation of the 491 bp dsDNA target with 70 nM YOYO-1 for 15 min resulted in a 68.4% decrease in fluorescence for the normalized 1 bp A-C mismatched DNA triplex (491 bp dsDNA+probe CF10) compared to that achieved with the normalized perfectly matched DNA triplex (491 bp dsDNA+probe CF01) (Table 8). When the pre-incubation of the 491 bp dsDNA target with 70 nM YOYO-1 was increased from 15 min to 3 hr in the absence of NaCl, the specificity of DNA triplex formation progressively decreased. The level of discrimination between normalized perfectly matched DNA triplex formation and normalized 1 bp A-C mismatched DNA triplex formation decreased from 68.4% to just 2.9% as the pre-incubation time of the 491 bp dsDNA target with 70 nM YOYO-1 increased from 15 min to 3 hr in the absence of NaCl (Table 8).

Pre-incubation of the ssDNA probes with 30 nM YOYO-1 for 3 hr together with pre-incubation of the 491 bp dsDNA target with 70 nM YOYO-1 for 15 min in the presence of 100 nM NaCl greatly increased both perfectly matched and 1 bp A-C mismatched DNA triplex formation. The level of discrimination between perfectly matched DNA triplex (491 bp dsDNA+probe CFO) formation and 1 bp A-C mismatched DNA triplex (491 bp dsDNA+probe CF10) formation was 61.2%, similar to that achieved in the absence of NaCl (Table 8). However, when the pre-incubation of the 491 bp dsDNA target with 70 nM YOYO-1 was increased from 15 min to 2 hr in the presence of 100 nM NaCl, the specificity of DNA triplex formation progressively increased, in sharp contrast to the progressive loss of specificity observed in the absence of NaCl. The fluorescent emission intensities achieved by the normalized 1 bp A-C mismatched DNA triplex (491 bp dsDNA+probe CF10) were 61.2%, 65.0%, 76.4% and 87.4% lower than those obtained from the normalized perfectly matched DNA triplex (491 bp dsDNA+probe CF01) after a 15 min, 30 min, 60 min and 120 min pre-incubation, respectively, in the presence of 100 nM NaCl (Table 8). The level of discrimination between perfectly matched DNA triplex formation and 1 bp A-C mismatched DNA triplex formation declined to only 65.1% following a 3 hr pre-incubation of the 491 bp dsDNA target with 70 nM YOYO-1 in the presence of 100 nM NaCl.

Therefore, the inclusion of 100 nM NaCl in extended duration pre-incubation of dsDNA target with 70 nM YOYO-1 combined with a 3 hr pre-incubation of ssDNA probes with 30 nM YOYO-1 results in greatly increased specificity of DNA triplex formation. The inclusion of 100 nM NaCl in short duration target pre-incubation can also positively affect the rate of binding and the assay's sensitivity.

### Example 6

This example demonstrates that DNA triplex binding specificity can be improved by electrical pretreatment of medium prior to its use to pre-incubate ssDNA probes with YOYO-1.

Aliquots of pre-incubation medium comprising 0.6×TBE, pH 8.3 either remained untreated or were electrically pretreated prior to addition of probe DNA. The medium was electrically pretreated by means of two platinum/iridium electrodes 2 mm apart, submerged in 66 μl of medium in a tube. Forty pulses of nine volts of DC current each with a duration of 500 msec and separated by 10 sec intervals were applied to the 66 μl of medium. Immediately after the final pulse of DC current, 1.25 pmole of wild-type or mutant ssDNA probe with 30 nM YOYO-1 were added to the untreated or electrically pretreated medium. Following a 1 hr incubation, 0.05 pmoles of PCR-amplified 491 bp dsDNA target (SEQ ID NO:1) and 70 nM YOYO-1 were added. The final buffer concentration was 0.5×TBE. The 80 μl reaction mixtures were then incubated for 5 minutes, placed into a quartz cuvette, irradiated with a 38 mW argon ion laser beam having a wavelength of 488 nm and monitored immediately for fluorescent emission. The laser irradiation period was 60 msec.

Pre-incubation of the control ssDNA probes with 30 nM YOYO-1 for 1 hour in the untreated medium produced noticeably less fluorescent emission intensity (Table 9). The fluorescent emission intensities emitted by a 1 bp A-C mismatched DNA triplex (491 bp dsDNA+probe CF10) and a 3 bp mismatched DNA triplex (491 bp dsDNA+probe CF508) were 57.3% and 45.3% lower, respectively, than that obtained by the perfectly matched DNA triplex (491 bp dsDNA+probe CF01), when normalized for variations in different ssDNA probe fluorescence (Table 9). The level of discrimination between perfectly complementary DNA triplex formation and incompletely complementary DNA triplex formation after a 1 hr unpretreated (i.e., no electrical pretreatment) pre-incubation of the ssDNA probes with 30 nM YOYO-1 was less than that achieved following a 2 hr pre-incubation of the ssDNA probes with 30 nM YOYO-1 (compare Table 3 and Table 9).

Electrical pretreatment of the pre-incubation medium prior to addition of DNA and YOYO-1 slightly increased the level of fluorescent emission intensity of each control ssDNA probe without affecting the fluorescent emission intensity of the control 491 bp dsDNA target (Table 9). Application of forty 9V pulses to the medium prior to addition and pre-incubation of the ssDNA probes with 30 nM YOYO-1 for 1 hour followed by addition of the 491 bp dsDNA target with 70 nM YOYO-1 significantly reduced both perfectly matched DNA triplex formation and mismatched DNA triplex formation. However, the specificity of mixed base DNA triplex formation was greatly improved as a consequence of application of forty 9V pulses to the medium used for probe pre-incubation. In the electrically pretreated probe pre-incubation medium, the fluorescent emission intensities achieved by a 1 bp A-C mismatched DNA triplex (491 bp dsDNA+probe CF10) and a 3 bp mismatched DNA triplex (491 bp dsDNA+probe CF508) were both 100% lower than that obtained by the perfectly matched DNA triplex (491 bp dsDNA+probe CF01), when normalized for variations in different ssDNA probe fluorescence (Table 9).

Electrical pretreatment of the pre-incubation medium prior to addition of DNA and YOYO-1 provides a means of minimizing the pre-incubation period for ssDNA probes with 30 nM YOYO-1 necessary to achieve maximum specificity in mixed base DNA triplex specificity, while resulting in a low level of ssDNA probe alone fluorescence. This protocol is also significant as the effect of electrical pretreatment of the medium is to improve triplex binding specificity. Furthermore electrical pretreatment effects demonstrated above are longer lived than previously observed in other experiments in which duplexes are formed.

### Example 7

This example demonstrates how DNA quadruplex binding specificity can be improved by electrical pretreatment of medium prior to its use to pre-incubate dsDNA probes with YOYO-1.

Human genomic dsDNA was extracted from clinical samples as described in Example 1. PCR amplification of wild-type homozygous (SEQ ID NO:1), mutant homozygous (SEQ ID NO:9) and mutant heterozygous (SEQ ID NO:10) dsDNA fragments of a region of exon 10 of the cystic fibrosis gene was performed as described in Example 1. The mutant homozygous PCR amplicons (SEQ ID NO:9) were homozygous for the cystic fibrosis A508 three base pair deletion at amino acid positions 507 and 508 at which the wild-type antisense sequence AAG is deleted. The mutant heterozygous PCR amplicon (SEQ ID NO:10) was heterozygous for this 3 bp deletion.

A sense 15-mer ssDNA sequence complementary to the antisense 15-mer ssDNA probe CF508 (SEQ ID NO:8) was synthesized, cartridge purified and dissolved in ddH₂O at a concentration of 1 pmole/μl as described in Example 1. Equimolar amounts of these complementary sense and antisense 15-mer ssDNA sequences were denatured at 95° C. for 10 minutes and allowed to anneal gradually in the presence of 10 mM Tris, pH 7.5, 1 mM EDTA and 100 mM NaCl, as the temperature cooled to 21° C. over 1.5 hours. The dsDNA probe produced (SEQ ID NO:11) was diluted in ddH₂O to a concentration of 1 pmole/μl. SEQ ID NO:11 was parallel homologous to a 15 bp region of the mutant homozygous PCR 491 bp dsDNA target (SEQ ID NO:9).

Sequence for the sense strand of the mutant 15-mer dsDNA probe (SEQ ID NO:11): 5′-AAT ATC ATT GGT GTT-3′.

Sequence for the antisense strand of the mutant 15-mer dsDNA probe (SEQ ID NO:11): 5′-AAC ACC AAT GAT ATT-3′.

Aliquots of pre-incubation medium comprising 0.6×TBE, pH 8.3 either remained untreated or were electrically pretreated prior to addition of probe DNA. Forty pulses of nine volts of DC current each with a duration of 500 msec and separated by 10 sec intervals were applied to the 66 μl of medium to be pretreated as described in Example 6. Immediately after the final pulse of DC current, 1.25 pmole of mutant dsDNA probe (SEQ ID NO:11) with 30 nM YOYO-1 were added to the untreated or electrically pretreated medium. Following a 1 hr incubation, 0.05 pmoles of wild-type or mutant PCR-amplified 491 bp dsDNA target and 70 nM YOYO-1 were added. The final buffer concentration of the reaction mixture was 0.5×TBE in a final volume of 80 μl. The final reaction mixtures were then incubated for 5 minutes, placed into a quartz cuvette, irradiated with a 38 mW argon ion laser beam having a wavelength of 488 nm and monitored immediately for fluorescent emission. The laser irradiation period was 60 msec.

When the mutant 15-mer dsDNA probe (SEQ ID NO:11) was reacted with the mutant 491 bp dsDNA targets (SEQ ID NO:9 and SEQ ID NO:10) or the wild-type 491 bp dsDNA target (SEQ ID NO:1), parallel homologous dsDNA:dsDNA complexes were formed in the untreated medium under non-denaturing conditions (Table 10). It was observed that parallel homologous quadruplexes, stabilized by YOYO-1 intercalation, formed more readily between a dsDNA target and a dsDNA probe when that probe contained perfectly homologous sequences, than when there was a 3 bp region which was not homologous, that is to say identical, to a 3 bp region in the dsDNA target. When the maximum fluorescent intensity values were normalized by subtracting the fluorescent intensity value obtained for the dsDNA probe control (SEQ ID NO:11), the mismatched quadruplexes comprised of SEQ ID NO:1+SEQ ID NO:11, emitted a fluorescent intensity that was 96.9% lower than that achieved by the perfectly homologous quadruplexes (SEQ ID NO:9+SEQ ID NO:11) in the untreated medium (Table 10). A heterozygous mix of mismatched quadruplexes and perfectly homologous quadruplexes (SEQ ID NO:10+SEQ ID NO:11) produced a fluorescent emission intensity that was 58.6% lower than that observed with the perfectly homologous quadruplexes (SEQ ID NO:9+SEQ ID NO:11) in the untreated medium (Table 10). The dsDNA target plus 100 nM YOYO-L control samples, which had been incubated for 5 minutes while the reaction mixtures were incubating for 5 minutes, showed levels of fluorescence, which constituted no more than 4.7% of the fluorescent level achieved by the perfectly matched quadruplex (Table 10).

Application of forty 9V pulses to the medium prior to addition and pre-incubation of the dsDNA probe CF508/C (SEQ ID NO:11) with 30 nM YOYO-1 increased the level of fluorescent emission of the control dsDNA probe without affecting the fluorescent emission intensities of the control 491 bp dsDNA targets (Table 10). Electrical pretreatment with forty 9V pulses to the pre-incubation medium prior to addition of probe DNA and YOYO-1 significantly increased perfectly homologous quadruplex (SEQ ID NO:9+SEQ ID NO:11) formation (Table 10), resulting in a 196% increase in fluorescent emission compared to that observed with the same sample in untreated test medium, when normalized for variations in dsDNA probe fluorescence in untreated and electrically pretreated medium (Table 10). The avidity or sensitivity of parallel homologous DNA quadruplex formation was greatly improved as a consequence of application of forty 9V pulses to the medium used for probe pre-incubation. When the maximum fluorescent intensity values were normalized by subtracting the fluorescent intensity value obtained for the dsDNA probe (SEQ ID NO:11), the mismatched quadruplexes comprised of SEQ ID NO:1+SEQ ID NO:11 emitted a fluorescent intensity that was 93.9% lower than that achieved by the perfectly homologous quadruplexes (SEQ ID NO:9+SEQ ID NO:11) in the pretreated medium (Table 10). This level of discrimination was nearly identical to those observed in fluorescent emissions from complexes formed in untreated medium. A heterozygous mix of mismatched quadruplexes and perfectly homologous quadruplexes (SEQ ID NO:10+SEQ ID NO:11) produced a fluorescent emission intensity that was 100% lower than that observed with the perfectly homologous quadruplexes (SEQ ID NO:9+SEQ ID NO:11) in the electrically pre-treated medium, representing a significant increase in specificity of parallel homologous DNA quadruplex formation as a result of electrical pretreatment of the medium used for probe pre-incubation (Table 10). Furthermore the electrical pretreatment effects demonstrated above are longer lived than the effects previously observed in some other similar experiments in which duplexes are formed.

### Example 8

This example demonstrates that antiparallel complementary DNA duplex specificity can be influenced by the pre-incubation of ssDNA probe with YOYO-1.

50-mer ssDNA target sequences, derived from exon 10 of the human cystic fibrosis gene [Nature 380, 207 (1996)] were synthesized on a DNA synthesizer, cartridge purified and dissolved in ddH₂O at a concentration of 1 pmole/μl.

Target JD123 (SEQ ID NO:12) was the sense strand of a 50-mer nucleotide segment of the wild-type PCR-amplified 491 bp dsDNA target (SEQ ID NO:1), and was completely complementary to the antisense probe CF01 (SEQ ID NO:4).

The sequence for target JD123 (SEQ ID NO:12) was: 5′-TGG CAC CAT TAA AGA AAA TAT CAT CTT TGG TGT TTC CTA TGA TGA ATA TA-3′

Table 11 shows the results when 1.25 pmoles of wild-type or mutant ssDNA probe were pre-incubated in a volume of 71.9 μl containing 0.5×TBE buffer with 30 nM YOYO-1 for 1 hr prior to the addition of 0.025 pmoles of 50-mer ssDNA target and 70 nM YOYO-1 in a volume of 8.1 μl. The 80 μl reaction mixtures were then incubated for 5 minutes, placed into a quartz cuvette, irradiated and monitored immediately for fluorescent emission.

Pre-incubation of the control ssDNA probes with 30 nM YOYO-1 for 1 hr significantly reduced the fluorescent emission intensity of each ssDNA probe (Table 11). The fluorescent emission intensities achieved by a 1 bp A-C mismatched DNA duplex (target JD123+probe CF10), a 1 bp T-C mismatched DNA duplex (target JD123+probe CF09), and a 1 bp T-G mismatched DNA duplex (target JD123+probe CF08), were 49.7%, 31.4% and 50.7% lower, respectively, than that obtained by the matched DNA duplex (target JD123+probe CF01), when normalized against the respective levels of pre-incubated ssDNA probe control fluorescence (Table 11). The difference in DNA duplex specificity observed depended on the particular ssDNA probe sequence used to form the DNA duplex.

Table 12 shows the results when 1.25 pmoles of wild-type or mutant ssDNA probe were pre-incubated in a volume of 73.15 μl containing 0.5×TBE buffer with 150 nM YOYO-1 for 1 hr prior to the addition of 1.25 pmoles of 50-mer ssDNA target and 350 nM YOYO-1 in a volume of 6.85 μl (samples 2-8). The final YOYO-1 concentration of the reaction mixture was 500 nM. For samples 10-16 of Table 12, all reagents were combined at the same time without pre-incubation. The 80 μl reaction mixtures were then incubated for 5 minutes, placed into Corning Non-binding Surface 384-well plates and irradiated with the GENEXUS ANALYZER 20 mW scanning solid state laser having a wavelength of 488 nm. 84 W/cm² of laser light is delivered to the samples from the bottom of each well. Irradiation occurred at a sampling interval of 60 microns at settings of 20 hertz, 42% PMT and 10 μA/V sensitivity. Fluorescent emission was monitored immediately.

Following pre-incubation of the ssDNA probes, incompletely complementary DNA duplexes containing a 1 bp A-C mismatch (target JD123+probe CF10) and a 1 bp T-C mismatch (target JD123+probe CF09) resulted in fluorescent emission intensities that were all 100% lower than those observed with the matched DNA duplexes (target JD123+probe CF01), when normalized for variations in different ssDNA probe fluorescence (Table 12). The variations in probe fluorescence were an expression of the degree of self-binding characteristic of each probe sequence. When all reagents were combined at the same time without pre-incubation, the fluorescent emission intensities achieved by a 1 bp A-C mismatched DNA duplex (target JD123+probe CF10) and a 1 bp T-C mismatched DNA duplex (target JD123+probe CF09) were 12.2% and 35.8% higher, respectively, than that obtained by the matched DNA duplex (target JD123+probe CF01), when normalized against the respective levels of ssDNA probe control fluorescence (Table 12). The specificity of antiparallel complementary duplex DNA formation was greatly increased by pre-incubating the probe with 150 nM YOYO-1 for 1 hr prior to duplex formation.

### Example 9

This example compares the level of DNA triplex specificity following pre-incubation that can be achieved wherein the mismatch site is internal or at either the 5′ or 3′ end of the triplex complex.

Antisense 15-mer ssDNA probe sequences, derived from exon 10 of the human cystic fibrosis gene were synthesized on a DNA synthesizer, cartridge purified and dissolved in ddH₂O at a concentration of 1 pmole/μl as described in Example 1.

Probe CF51 (SEQ ID NO:13) was a 15-mer ssDNA probe designed to be completely complementary to a 15 nucleotide segment of the sense strand of the wild-type PCR-amplified 491 bp dsDNA target (SEQ ID NO:1), except for a one base mismatch at the 5′ end (underlined). Probe 51 was identical in sequence to wild-type probe CF01, except for the one base mismatch at the 5′end.

The sequence for probe CF51 (SEQ ID NO:13) was: 5′-TAC CAA AGA TGA TAT-3′.

Probe CF31 was a 15-mer mutant ssDNA probe identical in sequence to wild-type probe CF01, except for a one base mismatch at the 3′ end (underlined).

The sequence for probe CF31 (SEQ ID NO:14) was: 5′-CAC CAA AGA TGA TAC-3′.

Table 13 shows the results when 1.25 pmoles of wild-type or mutant ssDNA probe were pre-incubated in 0.5×TBE buffer in presence of 75 nM NaCl for 1 hr followed by a further incubation in the presence of 30 nM YOYO-1 for 1 hr prior to the addition of 0.05 pmoles of PCR-amplified 491 bp dsDNA target (SEQ ID NO:1) and 70 nM YOYO-1 to form reaction mixtures. The 80 μl reaction mixtures were then incubated for 5 minutes, placed into a quartz cuvette, irradiated and monitored immediately for fluorescent emission.

Pre-incubation of the ssDNA probes in buffer containing 75 nM NaCl for 1 hr followed by a further 1 hr incubation after addition of 30 nM YOYO-1 resulted in a 100% decrease in fluorescence for both the 1 bp T-G mismatched DNA triplex (491 bp dsDNA+probe CF08) and the 1 bp G-T mismatched DNA triplex (491 bp dsDNA+probe CF51) compared to that achieved with the matched DNA triplex (491 bp dsDNA+probe CF01) (Table 13). These results demonstrate that the same high degree of DNA triplex specificity may be obtained when the 1 bp mismatch site is internal or at the 5′ end of the triplex complex following pre-incubation of the ssDNA probes in the presence of NaCl prior to addition to the reaction mixture.

The fluorescent emission intensities achieved by the 1 bp A-C mismatched DNA triplex (491 bp dsDNA+probe CF10) and the 1 bp A-C mismatched DNA triplex (491 bp dsDNA+probe CF31) in the presence of 75 nM NaCl were 46.2% and 73.8% lower, respectively, than that obtained by the matched DNA triplexes (491 bp dsDNA+probe CF01) at this NaCl concentration (Table 13). Positioning of the 1 bp A-C mismatch at the 3′ end of the triplex complex as opposed to an internal site resulted in a marked enhancement of specificity of DNA triplex formation when the above probe pre-incubation protocol was performed (Table 13).

Table 14 shows the results when 0.05 pmoles of PCR-amplified 491 bp dsDNA target (SEQ ID NO:1) were pre-incubated in 0.5×TBE buffer with 70 nM YOYO-1 in the presence of 50 mM NaCl for 15 min, with mixing steps at 7.5 min and 15 min, prior to the addition of 1.25 pmoles of wild-type or mutant ssDNA probe and 30 nM YOYO-1 to form reaction mixtures. The 80 μl reaction mixtures were then incubated for 5 minutes, placed into Corning Non-binding Surface 384-well plates and irradiated with the GENEXUS ANALYZER 20 mW scanning solid state laser having a wavelength of 488 nm. 84 W/cm² of laser light is delivered to the samples from the bottom of each well. Irradiation occurred at a sampling interval of 60 microns at settings of 20 hertz, 42% PMT and 10 μA/V sensitivity. Fluorescent emission was monitored immediately.

Inclusion of 50 mM NaCl during pre-incubation of the 491 bp dsDNA target with 70 nM YOYO-1 for 15 min, as well as in the control ssDNA probe samples resulted in relatively high levels of fluorescence emission in the control samples but good specificity of DNA triplex formation (Table 14), consistent with the results previously shown in Table 5. The fluorescent emission intensities achieved by the 1 bp T-G mismatched DNA triplex (491 bp dsDNA+probe CF08) and the 1 bp G-T mismatched DNA triplex (491 bp dsDNA+probe CF51) following pre-incubation of the 491 bp dsDNA target with 50 mM NaCl and 70 nM YOYO-1 were 62.5% and 100% lower, respectively, than that obtained by the matched DNA triplexes (491 bp dsDNA+probe CF01) (Table 14). Positioning of the 1 bp mismatch at the 5′ end of the triplex complex as opposed to an internal site resulted in a significant enhancement of specificity of DNA triplex formation when the above target pre-incubation protocol was performed (Table 14).

The difference in fluorescence emission between the matched DNA triplex and the 1 bp A-C mismatched DNA triplexes were very similar irrespective of the positioning of the mismatch site in the DNA triplex when the above target pre-incubation protocol was performed. Inclusion of 50 mM NaCl during pre-incubation of the 491 bp dsDNA target with 70 nM YOYO-1 for 15 min resulted in an 81.0% decrease and a 72.1% decrease in fluorescent emission intensities observed with the internal 1 bp A-C mismatched DNA triplex (491 bp dsDNA+probe CF10) and the 3′ end located 1 bp A-C mismatched DNA triplex (491 bp dsDNA+probe CF31), respectively, compared to that obtained with the matched DNA triplexes (491 bp dsDNA+probe CF01) (Table 14).

Positioning of 1 bp mismatches at either the 5′ end or 3′ end of triplex complexes as opposed to an internal site can result in either a significant enhancement of DNA triplex specificity or no loss in DNA triplex specificity. Pre-incubation of ssDNA probes or pre-incubation of dsDNA targets with specific pre-incubation agents can enhance specific triplex binding. Results are consistent with our heteropolymeric triplex nucleating at either the 3′ or 5′ end of the third strand. Maximum flexibility in triplex probe design is accordingly available.

While the invention has been described in detail and with reference to specific examples thereof, it will be apparent to one skilled in the art that various changes and modifications can be made therein without departing from the spirit and scope thereof.

TABLE 1

| | Fluorescence | | | Fluorescence | | |
|---|---|---|---|---|---|---|
| Sample | @ 250 msec | Minus | Percent | @ 250 msec | Minus | Percent |
| (Probe:target = 25:1) | after 5 min | ssDNA | decrease | after 90 min | ssDNA | decrease |
| 1) YOYO-1 (100 nM) | 174 | | | 274 | | |
| 2) 491 bp (0.05 pmole) | 449 | | | 1641 | | |
| 3) CF01 (1.25 pmole) | 2258 | 0 | | 2046 | 0 | |
| 4) CF01 + 491 bp (perfect | 3373 | 1115 | | 3939 | 1893 | |
| match) | | | | | | |
| 5) CF10 (1.25 pmole) | 2125 | 0 | | 2800 | 0 | |
| 6) CF10 + 491 bp (1 bp A-C) | 2570 | 445 | −60.1% | 1017 | <0 | −100% |
| 7) 491 bp (0.05 pmole) | 351 | | | 415 | | |
| 8) CF01 (1.25 pmole) | 1515 | 0 | | 1528 | 0 | |
| 9) CF01 + 491 bp (perfect | 2484 | 969 | | 2066 | 538 | |
| match) | | | | | | |
| 10) CF10 (1.25 pmole) | 1339 | 0 | | 964 | 0 | |
| 11) CF10 + 491 bp (1 bp A-C) | 1801 | 462 | −52.3% | 841 | <0 | −100% |
| 12) 491 bp (0.05 pmole) | 306 | | | 240 | | |
| 13) CF01 (1.25 pmole) | 1885 | 0 | | 1592 | 0 | |
| 14) CF01 + 491 bp (perfect | 2727 | 842 | | 2256 | 664 | |
| match) | | | | | | |
| 15) CF10 (1.25 pmole) | 1362 | 0 | | 607 | 0 | |
| 16) CF10 + 491 bp (1 bp A-C) | 1695 | 333 | −60.5% | 876 | 269 | −59.5% |

Notes:
Test CF triplex assays on argon ion laser (10 mW at sample).
Samples 2-6 have PCR dsDNA and/or ssDNA + 100 nM YOYO-1.
Samples 7-16 have dsDNA pre-incubated with YOYO-1 for 15 min prior to addition of ssDNA and YOYO-1.
Samples 7-11 have PCR dsDNA + 70 nM YOYO-1 and/or ssDNA + 30 nM YOYO-1.
Samples 12-16 have PCR dsDNA + 60 nM YOYO-1 and/or ssDNA + 40 nM YOYO-1.

TABLE 2

| | Fluorescence | | |
|---|---|---|---|
| Sample | @ 250 msec | Minus | Percent |
| (Probe:target = 25:1) | after 5 min | ssDNA | decrease |
| 1) YOYO-1 (100 nM) | 160 | | |
| 2) 491 bp (0.05 pmole) | 273 | | |
| 3) CF01 (1.25 pmole) | 2110 | 0 | |
| 4) CF01 + 491 bp (perfect match) | 3318 | 1208 | |
| 5) CF10 (1.25 pmole) | 2322 | 0 | |
| 6) CF10 + 491 bp (1 bp A-C) | 2082 | <0 | −100% |
| 7) CF09 (1.25 pmole) | 2203 | 0 | |
| 8) CF09 + 491 bp (1 bp T-C) | 2509 | 306 | −74.7% |
| 9) CF08 (1.25 pmole) | 2323 | 0 | |
| 10) CF08 + 491 bp (1 bp T-G) | 2647 | 324 | −73.2% |
| 11) CF508 (1.25 pmole) | 2307 | 0 | |
| 12) CF508 + 491 bp (3 bp) | 2825 | 518 | −57.1% |

Notes:
Pre-incubation of dsDNA target with 70 nM YOYO-1 for 15 min prior to addition of ssDNA probe and 30 nM YOYO-1.
Test CF triplex assays on argon ion laser (10 mW at sample).
Samples 2-12 have PCR dsDNA + 70 nM YOYO-1 and/or ssDNA + 30 nM YOYO-1.

TABLE 3

| | Fluorescence | | |
|---|---|---|---|
| Sample | @ 250 msec | Minus | Percent |
| (Probe:target = 25:1) | after 5 min | ssDNA | decrease |
| 1) YOYO-1 (100 nM) | 154 | | |
| 2) 491 bp (0.05 pmole) | 277 | | |
| 3) CF01 (1.25 pmole) | 461 | 0 | |
| 4) CF01 + 491 bp (perfect match) | 1337 | 876 | |
| 5) CF10 (1.25 pmole) | 343 | 0 | |
| 6) CF10 + 491 bp (1 bp A-C) | 636 | 293 | −66.5% |
| 7) CF09 (1.25 pmole) | 908 | 0 | |
| 8) CF09 + 491 bp (1 bp T-C) | 860 | <0 | −100% |
| 9) CF08 (1.25 pmole) | 875 | 0 | |
| 10) CF08 + 491 bp (1 bp T-G) | 1042 | 167 | −80.9% |
| 11) CF508 (1.25 pmole) | 919 | 0 | |
| 12) CF508 + 491 bp (3 bp) | 1033 | 114 | −87.0% |

Notes:
Pre-incubation of ssDNA probe with 30 nM YOYO-1 for 2 hr prior to addition of dsDNA and 70 nM YOYO-1.
Test CF triplex assays on argon ion laser (10 mW at sample).
Samples 2-12 have PCR dsDNA + 70 nM YOYO-1 and/or ssDNA + 30 nM YOYO-1.

TABLE 4

| | Fluorescence | | |
|---|---|---|---|
| Sample | @ 250 msec | Minus | Percent |
| (Probe:target = 25:1) | after 5 min | ssDNA | decrease |
| 1) YOYO-1 (100 nM) | 148 | | |
| 2) 491 bp (0.05 pmole) | 226 | | |
| 3) CF01 (1.25 pmole) | 511 | 0 | |
| 4) CF01 + 491 bp (perfect match) | 1081 | 570 | |
| 5) CF10 (1.25 pmole) | 276 | 0 | |
| 6) CF10 + 491 bp (1 bp A-C) | 406 | 130 | −77.2% |
| 7) CF09 (1.25 pmole) | 338 | 0 | |
| 8) CF09 + 491 bp (1 bp T-C) | 634 | 296 | −48.1% |
| 9) CF08 (1.25 pmole) | 540 | 0 | |
| 10) CF08 + 491 bp (1 bp T-G) | 631 | 91 | −84.0% |
| 11) CF508 (1.25 pmole) | 425 | 0 | |
| 12) CF508 + 491 bp (3 bp) | 525 | 100 | −82.5% |

Notes:
Pre-incubation of ssDNA probe with 30 nM YOYO-1 for 2 hr and pre-incubation of dsDNA target with 70 nM YOYO-1 for 15 min.
Test CF triplex assays on argon ion laser (10 mW at sample).
Samples 2-12 have PCR dsDNA + 70 nM YOYO-1 and/or ssDNA + 30 nM YOYO-1.

TABLE 5

| | | Fluorescence | | |
|---|---|---|---|---|
| Sample | | @ 250 msec | | Percent |
| (Probe:target = 25:1) | [NaCl] | after 5 min | Minus ssDNA | decrease |
| 1) YOYO-1 (100 nM) | 0 | 172 | | |
| 2) 491 bp (0.05 pmole) | 0 | 275 | | |
| 3) CF01 (1.25 pmole) | 0 | 2087 | 0 | |
| 4) CF01 + 491 bp (perfect match) | 0 | 3274 | 1187 | |
| 5) CF09 (1.25 pmole) | 0 | 2853 | 0 | |
| 6) CF09 + 491 bp (1 bp T-C) | 0 | 3565 | 712 | −40.0% |
| 7) 491 bp (0.05 pmole) | 50 nM | 275 | | |
| 8) CF01 (1.25 pmole) | 50 nM | 2861 | 0 | |
| 9) CF01 + 491 bp (perfect match) | 50 nM | 3737 | 876 | |
| 10) CF09 (1.25 pmole) | 50 nM | 2572 | 0 | |
| 11) CF09 + 491 bp (1 bp T-C) | 50 nM | 2935 | 363 | −58.6% |
| 12) 491 bp (0.05 pmole) | 75 nM | 275 | | |
| 13) CF01 (1.25 pmole) | 75 nM | 2517 | 0 | |
| 14) CF01 + 491 bp (perfect match) | 75 nM | 3331 | 814 | |
| 15) CF09 (1.25 pmole) | 75 nM | 2711 | 0 | |
| 16) CF09 + 491 bp (1 bp T-C) | 75 nM | 2534 | <0 | −100% |
| 17) 491 bp (0.05 pmole) | 50 mM | 275 | | |
| 18) CF01 (1.25 pmole) | 50 mM | 1912 | 0 | |
| 19) CF01 + 491 bp (perfect match) | 50 mM | 3053 | 1141 | |
| 20) CF09 (1.25 pmole) | 50 mM | 160 | 0 | |
| 21) CF09 + 491 bp (1 bp T-C) | 50 mM | 262 | 102 | −91.1% |

Notes:
Pre-incubation of dsDNA target ± NaCl with 70 nM YOYO-1 for 15 min prior to addition of ssDNA probe and 30 nM YOYO-1.
Test CF triplex assays on argon ion laser (10 mW at sample).
Samples 2-21 have PCR dsDNA + 70 nM YOYO-1 and/or ssDNA + 30 nM YOYO-1.

TABLE 6

| | | Fluorescence | | |
|---|---|---|---|---|
| Sample | [NaCl] | @ 40% PMT | | Percent |
| (Probe:target = 25:1) | (nM) | after 5 min | Minus ssDNA | decrease |
| 1) YOYO-1 (100 nM) | 0 | 0 | | |
| 2) 491 bp (0.05 pmole) | 0 | 1868 | | |
| 3) CF01 (1.25 pmole) | 0 | 3575 | 0 | |
| 4) CF01 + 491 bp (perfect match) | 0 | 16032 | 12457 | |
| 5) CF10 (1.25 pmole) | 0 | 0 | 0 | |
| 6) CF10 + 491 bp (1 bp A-C) | 0 | 6397 | 6397 | −48.6% |
| 7) CF01 (1.25 pmole) | 50 | 3614 | 0 | |
| 8) CF01 + 491 bp (perfect match) | 50 | 20105 | 16491 | |
| 9) CF10 (1.25 pmole) | 50 | 80 | 0 | |
| 10) CF10 + 491 bp (1 bp A-C) | 50 | 7673 | 7593 | −54.0% |
| 11) CF01 (1.25 pmole) | 75 | 3677 | 0 | |
| 12) CF01 + 491 bp (perfect match) | 75 | 21754 | 18077 | |
| 13) CF10 (1.25 pmole) | 75 | 8 | 0 | |
| 14) CF10 + 491 bp (1 bp A-C) | 75 | 8379 | 8371 | −53.7% |
| 15) CF01 (1.25 pmole) | 100 | 4684 | 0 | |
| 16) CF01 + 491 bp (perfect match) | 100 | 18855 | 14171 | |
| 17) CF10 (1.25 pmole) | 100 | 145 | 0 | |
| 18) CF10 + 491 bp (1 bp A-C) | 100 | 8539 | 8394 | −40.8% |

Notes:
Pre-incubation of ssDNA probe ± NaCl with 30 nM YOYO-1 for 2 hr prior to addition of dsDNA and 70 nM YOYO-1.
Test CF triplex assays on Genexus argon ion laser (10 mW at sample).
Samples 2-18 have PCR dsDNA + 70 nM YOYO-1 and/or ssDNA + 30 nM YOYO-1.

TABLE 7

| | | Fluorescence | | |
|---|---|---|---|---|
| Sample | [NaCl] | @ 40% PMT | | Percent |
| (Probe:target = 25:1) | (nM) | after 5 min | Minus ssDNA | decrease |
| 1) YOYO-1 (100 nM) | 0 | 0 | | |
| 2) 491 bp (0.05 pmole) | 0 | 1868 | | |
| 3) CF01 (1.25 pmole) | 0 | 0 | 0 | |
| 4) CF01 + 491 bp (perfect match) | 0 | 9804 | 9804 | |
| 5) CF10 (1.25 pmole) | 0 | 0 | 0 | |
| 6) CF10 + 491 bp (1 bp A-C) | 0 | 2485 | 2485 | −74.7% |
| 7) CF01 (1.25 pmole) | 50 | 0 | 0 | |
| 8) CF01 + 491 bp (perfect match) | 50 | 14700 | 14700 | |
| 9) CF10 (1.25 pmole) | 50 | 0 | 0 | |
| 10) CF10 + 491 bp (1 bp A-C) | 50 | 2423 | 2423 | −83.5% |
| 11) CF01 (1.25 pmole) | 75 | 0 | 0 | |
| 12) CF01 + 491 bp (perfect match) | 75 | 12977 | 12977 | |
| 13) CF10 (1.25 pmole) | 75 | 0 | 0 | |
| 14) CF10 + 491 bp (1 bp A-C) | 75 | 1702 | 1702 | −86.9% |
| 15) CF01 (1.25 pmole) | 100 | 0 | 0 | |
| 16) CF01 + 491 bp (perfect match) | 100 | 13635 | 13635 | |
| 17) CF10 (1.25 pmole) | 100 | 0 | 0 | |
| 18) CF10 + 491 bp (1 bp A-C) | 100 | 2432 | 2432 | −82.2% |

Notes:
Pre-incubation of ssDNA probe ± NaCl for 1 hr followed by further incubation with 30 nM YOYO-1 for 2 hr prior to addition of dsDNA and 70 nM YOYO-1.
Test CF triplex assays on Genexus argon ion laser (10 mW at sample).
Samples 2-18 have PCR dsDNA + 70 nM YOYO-1 and/or ssDNA + 30 nM YOYO-1.

TABLE 8

| | Time of | 0 nM NaCl | | | 100 nM NaCl | | |
|---|---|---|---|---|---|---|---|
| | dsDNA + | Fluorescence | | | Fluorescence | | |
| Sample | YOYO-1 | @ 40% PMT | Minus | Percent | @ 40% PMT | Minus | Percent |
| (Probe:target = 25:1) | (min) | after 5 min | ssDNA | decrease | after 5 min | ssDNA | decrease |
| 1) YOYO-1 (100 nM) | 15 | 506 | | | 216 | | |
| 2) 491 bp (0.05 pmole) | 15 | 1287 | | | 1293 | | |
| 3) CF01 (1.25 pmole) | 15 | 511 | 0 | | 750 | 0 | |
| 4) CF01 + 491 bp (perfect | 15 | 5060 | 4549 | | 11700 | 10950 | |
| match) | | | | | | | |
| 5) CF10 (1.25 pmole) | 15 | 531 | 0 | | 375 | 0 | |
| 6) CF10 + 491 bp (1 bp A-C) | 15 | 1968 | 1437 | −68.4% | 4622 | 4247 | −61.2% |
| 7) YOYO-1 (100 nM) | 30 | 424 | | | 316 | | |
| 8) 491 bp (0.05 pmole) | 30 | 2219 | | | 1738 | | |
| 9) CF01 (1.25 pmole) | 30 | 642 | 0 | | 927 | 0 | |
| 10) CF01 + 491 bp (perfect | 30 | 3907 | 3265 | | 10430 | 9503 | |
| match) | | | | | | | |
| 11) CF10 (1.25 pmole) | 30 | 658 | 0 | | 584 | 0 | |
| 12) CF10 + 491 bp (1 bp A-C) | 30 | 2267 | 1609 | −50.7% | 3911 | 3327 | −65.0% |
| 13) YOYO-1 (100 nM) | 60 | 450 | | | 350 | | |
| 14) 491 bp (0.05 pmole) | 60 | 1443 | | | 1367 | | |
| 15) CF01 (1.25 pmole) | 60 | 513 | 0 | | 1366 | 0 | |
| 16) CF01 + 491 bp (perfect | 60 | 2236 | 1723 | | 10188 | 8822 | |
| match) | | | | | | | |
| 17) CF10 (1.25 pmole) | 60 | 628 | 0 | | 434 | 0 | |
| 18) CF10 + 491 bp (1 bp A-C) | 60 | 1759 | 1131 | −34.4% | 2518 | 2084 | −76.4% |
| 19) YOYO-1 (100 nM) | 120 | 348 | | | 658 | | |
| 20) 491 bp (0.05 pmole) | 120 | 1600 | | | 2168 | | |
| 21) CF01 (1.25 pmole) | 120 | 595 | 0 | | 1455 | 0 | |
| 22) CF01 + 491 bp (perfect | 120 | 2104 | 1509 | | 8745 | 7290 | |
| match) | | | | | | | |
| 23) CF10 (1.25 pmole) | 120 | 551 | 0 | | 255 | 0 | |
| 24) CF10 + 491 bp (1 bp A-C) | 120 | 1777 | 1226 | −18.8% | 1176 | 921 | −87.4% |
| 25) YOYO-1 (100 nM) | 180 | 375 | | | 682 | | |
| 26) 491 bp (0.05 pmole) | 180 | 1062 | | | 2749 | | |
| 27) CF01 (1.25 pmole) | 180 | 618 | 0 | | 1359 | 0 | |
| 28) CF01 + 491 bp (perfect | 180 | 1420 | 802 | | 4175 | 2816 | |
| match) | | | | | | | |
| 29) CF10 (1.25 pmole) | 180 | 577 | 0 | | 332 | 0 | |
| 30) CF10 + 491 bp (1 bp A-C) | 180 | 1356 | 779 | −2.9% | 1316 | 984 | −65.1% |

Notes:
Pre-incubation of ssDNA probe with 30 nM YOYO-1 for 3 hr and pre-incubation of dsDNA target ± NaCl with 70 nM YOYO-1 for 15 min to 3 hr.
Test CF triplex assays on GENEXUS solid state laser (19 mW at sample).

TABLE 9

| | Frequency and | | | |
|---|---|---|---|---|
| | voltage of | Fluorescence | | |
| Sample | current applied | @ 60 msec | Minus | Percent |
| (Probe:target = 25:1) | to medium | after 5 min | ssDNA | decrease |
| 1) YOYO-1 (100 nM) | 0 | 141 | | |
| 2) 491 bp (0.05 pmole) | 0 | 191 | | |
| 3) CF01 (1.25 pmole) | 0 | 683 | 0 | |
| 4) CF01 + 491 bp (perfect match) | 0 | 2408 | 1725 | |
| 5) CF10 (1.25 pmole) | 0 | 207 | 0 | |
| 6) CF10 + 491 bp (1 bp A-C) | 0 | 944 | 737 | −57.3% |
| 7) CF508 (1.25 pmole) | 0 | 683 | 0 | |
| 8) CF508 + 491 bp (3 bp) | 0 | 1627 | 944 | −45.3% |
| 9) YOYO-1 (100 nM) | 40 × 9 V | 147 | | |
| 10) 491 bp (0.05 pmole) | 40 × 9 V | 211 | | |
| 11) CF01 (1.25 pmole) | 40 × 9 V | 795 | 0 | |
| 12) CF01 + 491 bp (perfect match) | 40 × 9 V | 1224 | 429 | |
| 13) CF10 (1.25 pmole) | 40 × 9 V | 520 | 0 | |
| 14) CF10 + 491 bp (1 bp A-C) | 40 × 9 V | 418 | <0 | −100% |
| 15) CF508 (1.25 pmole) | 40 × 9 V | 898 | 0 | |
| 16) CF508 + 491 bp (3 bp) | 40 × 9 V | 767 | <0 | −100% |

Notes:
Application of voltage (40 × 9 V) to medium prior to pre-incubation of ssDNA probe with 30 nM YOYO-1 for 1 hr followed by addition of dsDNA and 70 nM YOYO-1.
Test CF triplex assays on argon ion laser (38 mW at sample).
Samples 2-8 and 10-16 have PCR dsDNA + 70 nM YOYO-1 and/or ssDNA + 30 nM YOYO-1.

TABLE 10

| | Frequency | | | |
|---|---|---|---|---|
| | and | | | |
| | voltage of | | | |
| | current | Fluorescence | | |
| Sample | applied to | @ 60 msec | Minus | Percent |
| (Probe:target = 25:1) | medium | after 5 min | ssDNA | decrease |
| 1) YOYO-1 (100 nM) | 0 | 148 | | |
| 2) mutant homozygous 491 bp (0.05 pmole) | 0 | 202 | | |
| 3) mutant heterozygous 491 bp (0.05 pmole) | 0 | 189 | | |
| 4) wild-type 491 bp (0.05 pmole) | 0 | 209 | | |
| 5) dsDNA CF508/C (1.25 pmole) | 0 | 1309 | 0 | |
| 6) CF508/C + homozygous 491 bp (perfect match) | 0 | 1437 | 128 | |
| 7) CF508/C + heterozygous 491 bp | 0 | 1362 | 53 | −58.6% |
| 8) CF508/C + wild-type 491 bp (3 bp) | 0 | 1313 | 4 | −96.9% |
| 9) YOYO-1 (100 nM) | 40 × 9 V | 152 | | |
| 10) mutant homozygous 491 bp (0.05 pmole) | 40 × 9 V | 216 | | |
| 11) mutant heterozygous 491 bp (0.05 pmole) | 40 × 9 V | 201 | | |
| 12) wild-type 491 bp (0.05 pmole) | 40 × 9 V | 203 | | |
| 13) dsDNA CF508/C (1.25 pmole) | 40 × 9 V | 1472 | 0 | |
| 14) CF508/C + homozygous 491 bp (perfect match) | 40 × 9 V | 1851 | 379 | |
| 15) CF508/C + heterozygous 491 bp | 40 × 9 V | 1378 | <0 | −100% |
| 16) CF508/C + wild-type 491 bp (3 bp) | 40 × 9 V | 1495 | 23 | −93.9% |

Notes:
Application of voltage (40 × 9 V) to medium prior to pre-incubation of dsDNA probe with 30 nM YOYO-1 for 1 hr followed by addition of PCR dsDNA and 70 nM YOYO-1.
Test CF quadruplex assays on argon ion laser (38 mW at sample).
Samples 2-8 and 10-16 have PCR dsDNA + 70 nM YOYO-1 and/or 15-mer dsDNA + 30 nM YOYO-1.

TABLE 11

| | Fluorescence | Minus | |
|---|---|---|---|
| Sample | @ 30 msec | ssDNA | Percent |
| (Probe:target = 50:1) | after 5 min | probe | decrease |
| 1) YOYO-1 (100 nM) | 128 | | |
| 2) 50-mer JD123 (0.025 pmole) | 138 | | |
| 3) CF01 (1.25 pmole) | 500 | 0 | |
| 4) CF01 + JD123 (perfect | 2132 | 1632 | |
| match) | | | |
| 5) CF10 (1.25 pmole) | 291 | 0 | |
| 6) CF10 + JD123 (1 bp A-C) | 1112 | 821 | −49.7% |
| 7) CF09 (1.25 pmole) | 467 | 0 | |
| 8) CF09 + JD123 (1 bp T-C) | 1586 | 1119 | −31.4% |
| 9) CF08 (1.25 pmole) | 487 | 0 | |
| 10) CF08 + JD123 (1 bp T-G) | 1291 | 804 | −50.7% |

Notes:
Pre-incubation of 15-mer ssDNA probe with 30 nM YOYO-1 for 1 hr prior to addition of 50-mer ssDNA target and 70 nM YOYO-1.
Test CF duplex assays on argon ion laser (38 mW at sample).
Samples 2-10 have 50-mer ssDNA + 70 nM YOYO-1 and/or 15-mer ssDNA + 30 nM YOYO-1.

TABLE 12

| | Fluorescence | Minus | |
|---|---|---|---|
| Sample | @ 42% PMT | ssDNA | Percent |
| (Probe:target = 1:1) | after 5 min | probe | decrease |
| 1) YOYO-1 (500 nM) | 2754 | | |
| 2) 50-mer JD123 (1.25 pmole) | 6551 | | |
| 3) CF01 (1.25 pmole) | 6607 | 0 | |
| 4) CF01 + JD123 (perfect | 7071 | 464 | |
| match) | | | |
| 5) CF10 (1.25 pmole) | 8760 | 0 | |
| 6) CF10 + JD123 (1 bp A-C) | 6430 | <0 | −100% |
| 7) CF09 (1.25 pmole) | 7030 | 0 | |
| 8) CF09 + JD123 (1 bp T-C) | 6616 | <0 | −100% |
| 9) YOYO-1 (500 nM) | 2351 | | |
| 10) 50-mer JD123 (1.25 pmole) | 7616 | | |
| 11) CF01 (1.25 pmole) | 5072 | 0 | |
| 12) CF01 + JD123 (perfect | 8739 | 3667 | |
| match) | | | |
| 13) CF10 (1.25 pmole) | 4038 | 0 | |
| 14) CF10 + JD123 (1 bp A-C) | 8153 | 4115 | +12.2% |
| 15) CF09 (1.25 pmole) | 4652 | 0 | |
| 16) CF09 + JD123 (1 bp T-C) | 9631 | 4979 | +35.8% |

Notes:
Pre-incubation of 15-mer ssDNA probe with 150 nM YOYO-1 for 1 hr prior to addition of 50-mer ssDNA target and 350 nM YOYO-1.
Test CF duplex assays on Genexus solid state laser (19 mW at sample).
Samples 2-8 have 50-mer ssDNA + 350 nM YOYO-1 and/or 15-mer ssDNA + 150 nM YOYO-1
Samples 10-16 have 50-mer ssDNA and/or 15-mer ssDNA + 500 nM YOYO-1.

TABLE 13

| | | Fluorescence | | |
|---|---|---|---|---|
| Sample | [NaCl] | @ 60 msec | | Percent |
| (Probe:target = 25:1) | (nM) | after 5 min | Minus ssDNA | decrease |
| 1) YOYO-1 (100 nM) | 75 | 156 | | |
| 2) 491 bp (0.05 pmole) | 75 | 184 | | |
| 3) CF01 (1.25 pmole) | 75 | 747 | 0 | |
| 4) CF01 + 491 bp (perfect match) | 75 | 1358 | 611 | |
| 5) CF08 (1.25 pmole) | 75 | 980 | 0 | |
| 6) CF08 + 491 bp (1 bp T-G) | 75 | 858 | <0 | −100% |
| 7) CF51 (1.25 pmole) | 75 | 736 | 0 | |
| 8) CF51 + 491 bp (1 bp G-T) | 75 | 726 | <0 | −100% |
| 9) CF10 (1.25 pmole) | 75 | 496 | 0 | |
| 10) CF10 + 491 bp (1 bp A-C) | 75 | 825 | 329 | −46.2% |
| 11) CF31 (1.25 pmole) | 75 | 863 | 0 | |
| 12) CF31 + 491 bp (1 bp A-C) | 75 | 1023 | 160 | −73.8% |

Notes:
Pre-incubation of ssDNA probe ± NaCl for 1 hr followed by further incubation with 30 nM YOYO-1 for 1 hr prior to addition of dsDNA and 70 nM YOYO-1.
Test CF triplex assays on argon ion laser (38 mW at sample).
Samples 2-12 have PCR dsDNA + 70 nM YOYO-1 and/or ssDNA + 30 nM YOYO-1.

TABLE 14

| | | Fluorescence | | |
|---|---|---|---|---|
| Sample | [NaCl] | @ 60 msec | Minus | Percent |
| (Probe:target = 25:1) | (mM) | after 5 min | ssDNA | decrease |
| 1) YOYO-1 (100 nM) | 50 | 1963 | | |
| 2) 491 bp (0.05 pmole) | 50 | 6447 | | |
| 3) CF01 (1.25 pmole) | 50 | 13056 | 0 | |
| 4) CF01 + 491 bp | 50 | 22680 | 9624 | |
| (perfect match) | | | | |
| 5) CF08 (1.25 pmole) | 50 | 18310 | 0 | |
| 6) CF08 + 491 bp (1 bp | 50 | 21923 | 3613 | −62.5% |
| T-G) | | | | |
| 7) CF51 (1.25 pmole) | 50 | 19280 | 0 | |
| 8) CF51 + 491 bp (1 bp | 50 | 18076 | <0 | −100% |
| G-T) | | | | |
| 9) CF10 (1.25 pmole) | 50 | 10784 | 0 | |
| 10) CF10 + 491 bp (1 bp | 50 | 12609 | 1825 | −81.0% |
| A-C) | | | | |
| 11) CF31 (1.25 pmole) | 50 | 15494 | 0 | |
| 12) CF31 + 491 bp (1 bp | 50 | 18180 | 2687 | −72.1% |
| A-C) | | | | |

Notes:
Pre-incubation of dsDNA target + 50 mM NaCl with 70 nM YOYO-1 for 15 min prior to addition of ssDNA and 30 nM YOYO-1.
Test CF triplex assays on Genexus solid state laser (19 mW at sample).
Samples 2-12 have PCR dsDNA + 70 nM YOYO-1 and/or ssDNA + 30 nM YOYO-1.

What is claimed is:

1. A method for assaying a target, said method comprising:
providing a target composition comprising the target in a target medium, wherein the target contains a target sequence of nucleic acids or nucleic acid analogues;
providing a probe composition comprising a probe in a probe medium, wherein the probe contains a probe sequence of nucleic acids or nucleic acid analogues;
providing a hybridization mixture comprising the target composition and the probe composition;
incubating the hybridization mixture for an incubation period effective to bind the target sequence to the probe sequence to provide a complex, wherein the probe sequence is bonded to the target sequence by Watson-Crick complementary base interaction or by homologous base interaction; and
detecting a signal correlated with a binding affinity of the probe for the target to assay the target,
wherein: (a) the target composition further comprises at least one target incubation agent and the target composition is incubated prior to being provided in the hybridization mixture, such that discrimination of the signal from background signals is enhanced; and/or (b) the probe composition further comprises at least one probe incubation agent and the probe composition is incubated prior to being provided in the hybridization mixture, such that discrimination of the signal from background signals is enhanced.

2. The method of claim 1, wherein the target composition is incubated prior to being provided in the hybridization mixture and the probe composition is not incubated prior to being provided in the hybridization mixture.

3. The method of claim 2, wherein the target incubation agent comprises at least one of an intercalating agent and a metal cation.

4. The method of claim 3, wherein the target incubation agent comprises YOYO-1 provided in a YOYO-1:Target ratio from 5:1 to 1280:1 and/or Na⁺ provided in a Na⁺:Target ratio from 5:1 to 2,000,000:1.

5. The method of claim 4, wherein the target composition is incubated for about 5 minutes to about 25 minutes prior to being provided in the hybridization mixture.

6. The method of claim 1, wherein the probe composition is incubated prior to being provided in the hybridization mixture and the target composition is not incubated prior to being provided in the hybridization mixture.

7. The method of claim 6, wherein the probe incubation agent comprises at least one of an intercalating agent and a metal cation.

8. The method of claim 7, wherein the probe incubation agent comprises YOYO-1 provided in a YOYO-1:Probe ratio from 0.25:1 to 100:1 and/or Na⁺ provided in a Na⁺:Probe ratio from 5:1 to 2000:1.

9. The method of claim 8, wherein the probe composition is incubated for about 1 hour to about 3 hours prior to being provided in the hybridization mixture.

10. The method of claim 1, further comprising applying electric voltage to the probe medium, the target medium or the hybridization mixture, wherein the electric voltage is applied in an amount such that discrimination of the signal from background signals is further enhanced.

11. The method of claim 10, wherein the electric voltage comprises a plurality of pulses having a voltage of about 9 volts each.

12. The method of claim 1, wherein the target composition and the probe composition are incubated prior to being provided in the hybridization mixture.

13. The method of claim 12, wherein the probe incubation agent and the target incubation agent are independently selected from the group consisting of an intercalating agent and a metal cation.

14. The method of claim 13, wherein the probe incubation agent comprises YOYO-1 provided in a YOYO-1:Probe ratio from 0.25:1 to 100:1 and/or Na⁺ provided in a Na⁺:Probe ratio from 5:1 to 2000:1 and the target incubation agent comprises YOYO-1 provided in a YOYO-1:Target ratio from 5:1 to 1280:1 and/or Na⁺ provided in a Na⁺:Target ratio from 5:1 to 2,000,000:1.

15. The method of claim 14, wherein the probe composition and the target composition are incubated for about 5 minutes to about 3 hours prior to being provided in the hybridization mixture.

16. The method of claim 1, wherein the probe contains a heteropolymeric probe sequence, the target contains a heteropolymeric target sequence, and the probe is bonded to the target by bonding of the heteropolymeric probe sequence to the heteropolymeric target sequence.

17. The method of claim 16, wherein the complex is a duplex, a triplex or a quadruplex.

18. The method of claim 17, wherein: (i) the complex is a duplex wherein the heteropolymeric probe sequence is bonded to the heteropolymeric target sequence by homologous base interaction with parallel or antiparallel directionality; or (ii) the complex is a duplex wherein the heteropolymeric probe sequence is bonded to the heteropolymeric target sequence by Watson-Crick complementary base interaction with parallel directionality.

19. The method of claim 17, wherein the complex is a duplex, and the heteropolymeric probe sequence is bonded to the heteropolymeric target sequence by Watson-Crick complementary base interaction with parallel or antiparallel directionality.

20. The method of claim 17, wherein the complex is a triplex.

21. The method of claim 17, wherein the complex is a quadruplex.

22. The method of claim 17, wherein the signal is fluorescence emitted by at least one label covalently bound to the probe.

23. The method of claim 17, wherein the signal is fluorescence emitted by at least one label non-covalently associated with the complex.

24. The method of claim 17, wherein a match or a mismatch between bases of the heteropolymeric probe sequence and bases of the heteropolymeric target sequence is detected.

25. The method of claim 17, wherein the probe or the target is covalently bound to a support, surface or semi-permeable membrane.

26. The method of claim 17, wherein the hybridization mixture further comprises at least one binding promoter selected from the group consisting of YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, cyanine monomers, ethidium bromide, ethidium homodimer-1, ethidium homodimer-2, ethidium derivatives, acridine, acridine orange, acridine derivatives, ethidium-acridine heterodimer, ethidium monoazide, propidium iodide, SYTO dyes, SYBR Green 1, SYBR dyes, Pico Green, SYTOX dyes and 7-aminoactinomycin D.

27. The method of claim 1, wherein the discrimination of the signal from background signals is enhanced by: (a) increasing binding affinity or signal strength of perfectly matched target and probe; and/or (b) decreasing binding affinity or signal strength of mismatched target and probe.

28. A kit for performing the method of claim 1, said kit comprising the probe, a label adapted to emit the signal, and at least one of the target incubation agent and the probe incubation agent.
